SEQUENCE LISTING (21 sequences)

SEQ ID NO: 1 - protein, 24 aa, Homo sapiens
MAPRGFSCLLLLTSEIDLPVKRRA

SEQ ID NO: 2 - protein, 24 aa, Artificial Sequence
  Synthesized
MAPRGFSCLLLLTGEIDLPVKRRA

SEQ ID NO: 3 - protein, 24 aa, Artificial Sequence
  Synthesized
MAPRGFSCLLLLTSEIDLPVKRRA

SEQ ID NO: 4 - protein, 24 aa, Artificial Sequence
  Synthesized
MAPAGASCLLLLTGEIDLPVKRRA

SEQ ID NO: 5 - protein, 25 aa, Artificial Sequence
  Synthesized
MAPAGASCLLLLTDSEIDLPVKRRA

SEQ ID NO: 6 - protein, 18 aa, Artificial Sequence
  Synthesized
PAGASCLLLLTDSEIDLP

SEQ ID NO: 7 - protein, 17 aa, Artificial Sequence
  Synthesized
PAGASRLLLLTGEIDLP

SEQ ID NO: 8 - protein, 32 aa, Artificial Sequence
  Synthesized
EFLIVIKSMAPRGFSCLLLLTSEIDLPVKRRA

SEQ ID NO: 9 - protein, 32 aa, Artificial Sequence
  Synthesized
EFLIVIKSMAPRGFSCLLLLTGEIDLPVKRRA

SEQ ID NO: 10 - protein, 32 aa, Artificial Sequence
  Synthesized
EFLIVIKSMAPAGASCLLLLTGEIDLPVKRRA

SEQ ID NO: 11 - protein, 26 aa, Artificial Sequence
  Synthesized
SALLRSIPAPAGASRLLLLTGEIDLP

SEQ ID NO: 12 - protein, 24 aa, Artificial Sequence
  Synthesized
MAPRGFSCRLLLTSEIDLPVKRRA

SEQ ID NO: 13 - protein, 24 aa, Artificial Sequence
  Synthesized
MTPRGFSCLLLPTSETDLPVKRRX

SEQ ID NO: 14 - protein, 24 aa, Artificial Sequence
  Synthesized
MAPRGFSCLLLSTSEIDLPVKRXX

SEQ ID NO: 15 - protein, 24 aa, Artificial Sequence
  Synthesized
MAPRGFSCLLLSTSEIDLPVKRRA

SEQ ID NO: 16 - protein, 24 aa, Artificial Sequence
  Synthesized
MCHWAGGASNTGDARGDVFGKQAG

SEQ ID NO: 17 - protein, 26 aa, Artificial Sequence
  Synthesized
MGVKFFTLSTRFFPSVQRAVPLWTNS

SEQ ID NO: 18 - protein, 38 aa, Artificial Sequence
  Synthesized
MLGYNFSSFPCGTISIAPGFNFYRLYFIWVNGLAKVVW

SEQ ID NO: 19 - protein, 26 aa, Artificial Sequence
  Synthesized
MLEVMFLVNRRGKICRVPFTFFNLSL

SEQ ID NO: 20 - protein, 24 aa, Artificial Sequence
  Synthesized
MYCSEVGFCSEVAPTEIFNAGLVV

SEQ ID NO: 21 - protein, 20 aa, Artificial Sequence
  Synthesized
MLDQDIPMVQPLLKVRLFND

(19) United States
(12) Patent Application Publication
(10) Pub. No.: US 2013/0053323 A1
(43) Pub. Date: Feb. 28, 2013

(54) METHODS AND USE RELATED TO HUMANIN AND HUMANIN-LIKE PEPTIDES

(71) Applicants: Emma Eriksson, Stockholm (SE); Lars Sävendahl, Stocksund (SE); Farasat Zaman, Huddinge (SE)

(72) Inventors: Emma Eriksson, Stockholm (SE); Lars Sävendahl, Stocksund (SE); Farasat Zaman, Huddinge (SE)

(21) Appl. No.: 13/515,346

(22) PCT Filed: Dec. 22, 2010
(86) PCT No.: PCT/EP2010/070564

§ 371 Date: Jun. 12, 2012

(30) Foreign Application Priority Data
Dec. 22, 2009 (SE) 0901607-2

(51) Int. Cl. A61K 38/17; C07K 7/08; A61P 19/00; C07K 14/435; A61K 38/10; A61P 19/10

(52) U.S. Cl. 514/16.9; 530/325; 530/324; 530/326

(57) ABSTRACT

The field of medical therapeutics, and more particularly to therapeutic peptides selectively active in the treatment of bone- or cartilage disorders. Provided herein are a series of humanin and humaninlike peptides, fragments, analogs, derivatives, and variants thereof, which have beneficial activities on cartilage tissues and/or bone tissues including the prevention of negative effects of drugs on cartilage tissues and/or bone tissues.

### FIELD OF THE INVENTION

The present invention relates to the field of medical therapeutics, and more particularly to therapeutic peptides selectively active in the treatment of bone- or cartilage disorders. Provided herein are a series of humanin and humanin-like peptides, fragments, analogs, derivatives, and variants thereof, which have beneficial activities on cartilage tissues and/or bone tissues including the prevention of negative effects of disorders/drugs on cartilage tissues and/or bone tissues by treatment with for example, but not limited to, glucocorticoids and/or chemotherapy.

### BACKGROUND OF THE INVENTION

Longitudinal bone growth occurs in the growth plate through a process called endochondral bone formation, .a process where resting zone chondrocytes are recruited to start active proliferation and then undergo differentiation, followed by apoptosis and later mineralization. The balance between proliferation and differentiation is a crucial regulatory step controlled by various growth factors/hormones acting in both endocrine and paracrine/autocrine ways. Any disturbance/alteration in these hormones systemically and/or locally within growth plate cartilage may lead to abnormal chondrogensis causing growth failure of long bones (Savendahl 2005).

Glucocorticoids (GCs) are widely used as anti-inflammatory and immunosuppressive drugs both in children and adults with chronic diseases. However, recent studies have revealed that long-term high-dose GC treatment often leads to growth failure in treated children, an effect which has been shown to be mediated through a negative effect on growth plate chondrocytes (Baron, Huang et al. 1992) (Loeb 1976). Furthermore, long-term use of GCs in adults (men and women) has also been reported to cause osteoporosis/bone fractures as a negative side effect. Dexamethasone (Dexa) is a widely used glucocortcoid used in children and adults to treat different diseases such as, asthma, inflammation and cancer. Beside the positive effects of Dexa on inflammation and cancer, its long-term use has been linked to negative side effects on bone. Previous studies (pre-clinical and clinical) show that long-term use of Dexa can cause bone growth retardation in children and osteoporosis both in children and adults (Demoly 2008). It is well known that Dexa can alter the processes of proliferation and differentiation in chondrocytes and other cells. Therefore, identification of new molecules/analogs/peptides with the potential to be used in combination with existing widely used drugs (such as glucocorticoids and other chemotherapeutic drugs) without altering actual effects of these drugs, but blocking the negative side effects is important for the development of new treatment strategies in different disorders.

During the past decades, the incidence of childhood cancers has increased (Mangano 1999). Moreover, the results of the cancer therapy in children and adolescents have improved and the number of childhood cancer survivors is increasing (Linet, Ries et al. 1999). However, there has been a significant increase in both acute and chronic toxicity associated with the more successful, but now highly intensive chemotherapy (CT) regimens (Linet, Ries et al. 1999). In view of the fact that the incidence of childhood cancers coincides with periods of rapid skeletal development, it has become particular important to understand the adverse effects of this modality of treatment (Pinkerton 1992). Disruption of the physiological cellular homeostasis of growth plate chondrocytes and/or bone cells results in skeletal growth disturbances. Consequently, numerous skeletal complications including short stature and osteoporosis are important long-term problems in adult cancer survivors.

The most commonly used CT drugs to treat childhood cancers include alkalyting agents, antimetabolites, antibiotics, plant alkaloids (Balis 1997) alone or in combination with other drugs. Studies from in vitro experiments demonstrated direct effects on chondrocyte proliferation after treatment with various DNA-damaging CT drugs (cisplatin, etoposide, carboplatin and actinomycin) causing irreversible cell loss (Robson, Anderson et al. 1998). In vivo studies in male Wistar rats reported reduction in tibia length of 18% and 5% when animals received doxorubicin and methotrexate respectively (van Leeuwen, Kamps et al. 2000). In addition, several studies showed that CT alone is responsible for the decline in height seen during treatment for acute lymhoblastic leukemia (Halton, Atkinson et al. 1998) (Shalet and Price 1981) (Clayton, Shalet et al. 1988). Similarly, the anti-metabolite 5-fluorouracil (5-FU) is used in the treatment of solid tumors not only in adult cancer patients but also in childhood solid tumors, hepatoblastoma, head and neck cancers, nasopharyngeal carcinoma, esophageal/gastric junction adenocarcinoma, glaucoma and skin cancer. In pre-clinical studies 5-FU has been reported to suppress bone growth, an effect associated with rapid and significant suppression of cell proliferation in the growth plate and metaphysis and induction of apoptosis in the metaphysis (Xian, Howarth et al. 2004). Other chemotherapeutic drugs such as etoposide and cyclophosphamide, administered alone or in combination, have been reported to damage the growth plate by causing structural and cellular changes in the growth plate cartilage (Xian, Cool et al. 2007). Several clinical studies have shown a reduced bone mineral density after treatment with different CTs for different types of cancers (Arikoski, Komulainen et al. 1999) (Arikoski, Komulainen et al. 1999) (Arikoski, Kroger et al. 1999). The reduced bone mineral density after CT treatment may be permanent and significantly influences fracture risk later in life.

The proteasome inhibitors (PIs), including bortezomib (Velcade™, PS-341), belong to another novel and promising group of CT that has past phase I clinical trials (Blaney, Bernstein et al. 2004) and are now under phase II clinical trials in pediatric cancers. However, secondary effects on normal bystander tissues of primary life saving modalities are so far unknown. Preliminary results from us indicates that PIs, such as MG262 and bortezomib targets essential cell populations within the growth plate causing permanent growth retardation and chondrocyte cell death, both in vitro and in vivo (Zaman, Menendez-Benito et al. 2007).

Hashimoto and colleagues in 2001 identified humanin (HN), a novel 24 amino acid peptide (Hashimoto, Ito et al. 2001). In fact HN, encoded from the 16S ribosomal RNA of the mitochondrial DNA, was first identified in surviving neurons from an Alzheimer's disease (AD) patient, and described as a potent neurosurvival factor (Hashimoto, Niikura et al. 2001). Since then, its protective role has been described not only from various AD related insults, but also against prion-induced (Sponne, Fifre et al. 2004) and chemical-induced damage (Mamiya and Ukai 2001), thus broadening its role as a neuroprotective factor. Subsequently, it has been shown to be protective against many other cytotoxic agents (Kariya, Takahashi et al. 2003) and also protect non-neuronal cells such as smooth muscle cells (Jung and Van Nostrand 2003), rat pheochromocytoma cells (Kariya, Takahashi et al. 2002) and lymphocytes (Kariya, Takahashi et al. 2003). The HN cDNA shares complete identity with the mitochondrial 16SrRNA gene but spans only about half the length of the ribosomal RNA. HN is both an intracellular and secreted protein. Its transcripts of mitochondrial origin has been detected in normal mouse testis and colon (by immunoblot and immunohistochemical analyses) using specific antibodies against HN peptide (Kato, Iwamoto et al. 2003) and is present in cerebrospinal fluid (CSF), seminal fluid and serum. HN levels in CSF are few orders of magnitude higher than that in circulation, also present in kidney, brain, and the gastrointestinal tract. Interestingly, HN is highly conserved along evolution among species (between 90-100% homology), e.g. plants, nematodes, rats, mice, human and many other species (Guo, Zhai et al. 2003).

So far, little has been discovered about the regulation of its production. HN has also been reported as an antagonist of Bax and Bid that induces survival in cancer cells (Guo, Zhai et al. 2003), cell survival by binding to putative cell-surface receptors (Ying, Iribarren et al. 2004) and as an IGFBP-3 partner that antagonizes the apoptotic actions of IGFBP-3 on cancer cells (Ikonen, Liu et al. 2003). Furthermore, IGFBP-3 is one of a number of peptides including insulin, leptin, adiponectin, and resistin that have been shown to act in the central nervous system to regulate glucose metabolism (Muse, Lam et al. 2007) (Obici, Zhang et al. 2002). Unlike these before mentioned peptides, IGFBP-3 is an HN partner that has pro-diabetogenic hypothalamic actions that are modulated by IGF-I (Muzumdar and Rao 2006). Finally, HN has been reported as a wide spectrum survival factor (Nishimoto, Matsuoka et al. 2004), but its exact mechanism of action remains unclear.

There are several analogs/derivatives/variants of HN with enhanced potency and stability that have been described, including humanin-Gly14 (HNG) (Hashimoto, Niikura et al. 2001) (Terashita, Hashimoto et al. 2003), HNG-F6A (non-IGFBP-3 binding) (Ikonen, Liu et al. 2003) and colivelin (hybrid peptide containing partial sequences of HN and ADNF9) (Chiba, Yamada et al. 2005). Interestingly, all these different analogs exert similar neuroprotective effects, but they differ in their potency. HN and its analogs and derivatives have shown therapeutic potential for an array of diseases including Alzheimer's disease (AD), diabetes and kidney failure (Matsuoka and Hashimoto 2010) (Matsuoka 2009) (Xu, Chua et al. 2006) (Hoang, Park et al. 2010) (Singh and Mascarenhas 2008).

### REFERENCES

Arikoski, P., J. Komulainen, et al. (1999). “Reduced bone density at completion of chemotherapy for a malignancy.” Arch Dis Child 80(2): 143-148.

Arikoski, P., J. Komulainen, et al. (1999). “Impaired development of bone mineral density during chemotherapy: a prospective analysis of 46 children newly diagnosed with cancer.” J Bone Miner Res 14(12): 2002-2009.

Arikoski, P., H. Kroger, et al. (1999). “Disturbance in bone turnover in children with a malignancy at completion of chemotherapy.” Med Pediatr Oncol 33(5): 455-461.

Balis, F. M. (1997). “The Challenge of Developing New Therapies for Childhood Cancers.” Oncologist 2(1): I-II.

Baron, J., Z. Huang, et al. (1992). “Dexamethasone acts locally to inhibit longitudinal bone growth in rabbits.” Am J Physiol 263(3 Pt 1): E489-492.

Blaney, S. M., M. Bernstein, et al. (2004). “Phase I study of the proteasome inhibitor bortezomib in pediatric patients with refractory solid tumors: a Children's Oncology Group study (ADVL0015).” J Clin Oncol 22(23): 4804-4809.

Chiba, T., M. Yamada, et al. (2005). “Development of a femtomolar-acting humanin derivative named colivelin by attaching activity-dependent neurotrophic factor to its N terminus: characterization of colivelin-mediated neuroprotection against Alzheimer's disease-relevant insults in vitro and in vivo.” J Neurosci 25(44): 10252-10261.

Clayton, P. E., S. M. Shalet, et al. (1988). “Growth in children treated for acute lymphoblastic leukaemia.” Lancet 1(8583): 460-462.

De Luca, F., K. M. Barnes, et al. (2001). “Regulation of growth plate chondrogenesis by bone morphogenetic protein-2. ” Endocrinology 142(1): 430-436.

Demoly, P. (2008). “Safety of intranasal corticosteroids in acute rhinosinusitis.” Am J Otolaryngol 29(6): 403-413.

Guo, B., D. Zhai, et al. (2003). “Humanin peptide suppresses apoptosis by interfering with Bax activation.” Nature 423(6938): 456-461.

Halton, J. M., S. A. Atkinson, et al. (1998). “Growth and body composition in response to chemotherapy in children with acute lymphoblastic leukemia.” Int J Cancer Suppl 11:81-84.

Hashimoto, Y., Y. Ito, et al. (2001). “Mechanisms of neuroprotection by a novel rescue factor humanin from Swedish mutant amyloid precursor protein.” Biochem Biophys Res Commun 283(2): 460-468.

Hashimoto, Y., T. Niikura, et al. (2001). “Detailed characterization of neuroprotection by a rescue factor humanin against various Alzheimer's disease-relevant insults.” J Neurosci 21(23): 9235-9245.

Hashimoto, Y., T. Niikura, et al. (2001). “A rescue factor abolishing neuronal cell death by a wide spectrum of familial Alzheimer's disease genes and Abeta.” Proc Natl Acad Sci USA 98(11): 6336-6341.

Hoang, P. T., P. Park, et al. (2010). “The neurosurvival factor Humanin inhibits beta-cell apoptosis via signal transducer and activator of transcription 3 activation and delays and ameliorates diabetes in nonobese diabetic mice.” Metabolism 59(3): 343-349.

Ikonen, M., B. Liu, et al. (2003). “Interaction between the Alzheimer's survival peptide humanin and insulin-like growth factor-binding protein 3 regulates cell survival and apoptosis.” Proc Natl Acad Sci USA 100(22): 13042-13047.

Jung, S. S. and W. E. Van Nostrand (2003). “Humanin rescues human cerebrovascular smooth muscle cells from Abeta-induced toxicity.” J Neurochem 84(2): 266-272.

Kariya, S., N. Takahashi, et al. (2003). “Humanin improves impaired metabolic activity and prolongs survival of serum-deprived human lymphocytes.” Mol Cell Biochem 254(1-2): 83-89.

Kariya, S., N. Takahashi, et al. (2002). “Humanin inhibits cell death of serum-deprived PC12h cells.” Neuroreport 13(6): 903-907.

Kato, T., K. Iwamoto, et al. (2003). “No association of mutations and mRNA expression of WFS1/wolframin with bipolar disorder in humans.” Neurosci Lett 338(1): 21-24.

Linet, M. S., L. A. Ries, et al. (1999). “Cancer surveillance series: recent trends in childhood cancer incidence and mortality in the United States.” J Natl Cancer Inst 91(12): 1051-1058.

Loeb, J. N. (1976). “Corticosteroids and growth.” N Engl J Med 295(10): 547-552.

Mamiya, T. and M. Ukai (2001). “[Gly(14)]-Humanin improved the learning and memory impairment induced by scopolamine in vivo.” Br J Pharmacol 134(8): 1597-1599.

Mangano, J. J. (1999). “A rise in the incidence of childhood cancer in the United States.” Int J Health Sery 29(2): 393-408.

Matsuoka, M. (2009). “Humanin; a defender against Alzheimer's disease?” Recent Pat CNS Drug Discov 4(1): 37-42.

Matsuoka, M. and Y. Hashimoto (2010). “Humanin and the receptors for humanin.” Mol Neurobiol 41(1): 22-28.

Muse, E. D., T. K. Lam, et al. (2007). “Hypothalamic resistin induces hepatic insulin resistance.” J Clin Invest 117(6): 1670-1678.

Muzumdar, H. and M. Rao (2006). “Pulmonary dysfunction and sleep apnea in morbid obesity.” Pediatr Endocrinol Rev 3 Suppl 4: 579-583.

Nishimoto, I., M. Matsuoka, et al. (2004). “Unravelling the role of Humanin.” Trends Mol Med 10(3): 102-105.

Obici, S., B. B. Zhang, et al. (2002). “Hypothalamic insulin signaling is required for inhibition of glucose production.” Nat Med 8(12): 1376-1382.

Owen, H. C., J. N. Miner, et al. (2007). “The growth plate sparing effects of the selective glucocorticoid receptor modulator, AL-438.” Mol Cell Endocrinol 264(1-2): 164-170.

Pinkerton, C. R. (1992). “Avoiding chemotherapy related late effects in children with curable tumours.” Arch Dis Child 67(9): 1116-1119.

Robson, H., E. Anderson, et al. (1998). “Chemotherapeutic agents used in the treatment of childhood malignancies have direct effects on growth plate chondrocyte proliferation.” J Endocrinol 157(2): 225-235.

Savendahl, L. (2005). “Hormonal regulation of growth plate cartilage.” Horm Res 64 Suppl 2: 94-97.

Shalet, S. M. and D. A. Price (1981). “Effect of treatment of malignant disease on growth in children.” Arch Dis Child 56(3): 235-236.

Singh, B. K. and D. D. Mascarenhas (2008). “Bioactive peptides control receptor for advanced glycated end product-induced elevation of kidney insulin receptor substrate 2 and reduce albuminuria in diabetic mice.” Am J Nephrol 28(6): 890-899.

Sponne, I., A. Fifre, et al. (2004). “Humanin rescues cortical neurons from prion-peptide-induced apoptosis.” Mol Cell Neurosci 25(1): 95-102.

Terashita, K., Y. Hashimoto, et al. (2003). “Two serine residues distinctly regulate the rescue function of Humanin, an inhibiting factor of Alzheimer's disease-related neurotoxicity: functional potentiation by isomerization and dimerization.” J Neurochem 85(6): 1521-1538.

van Leeuwen, B. L., W. A. Kamps, et al. (2000). “Effect of single chemotherapeutic agents on the growing skeleton of the rat.” Ann Oncol 11(9): 1121-1126.

Wassberg, E., S. Pahiman, et al. (1997). “The angiogenesis inhibitor TNP-470 reduces the growth rate of human neuroblastoma in nude rats.” Pediatr Res 41(3): 327-333.

Xian, C. J., J. C. Cool, et al. (2007). “Cellular mechanisms for methotrexate chemotherapy-induced bone growth defects.” Bone 41(5): 842-850.

Xian, C. J., G. S. Howarth, et al. (2004). “Effects of acute 5-fluorouracil chemotherapy and insulin-like growth factor-I pretreatment on growth plate cartilage and metaphyseal bone in rats.” Bone 35(3): 739-749.

Xu, X., C. C. Chua, et al. (2006). “Humanin is a novel neuroprotective agent against stroke.” Stroke 37(10): 2613-2619.

Ying, G., P. Iribarren, et al. (2004). “Humanin, a newly identified neuroprotective factor, uses the G protein-coupled formylpeptide receptor-like-1 as a functional receptor.” J Immunol 172(11): 7078-7085.

Zaman, F., V. Menendez-Benito, et al. (2007). “Proteasome inhibition up-regulates p53 and apoptosis-inducing factor in chondrocytes causing severe growth retardation in mice.” Cancer Res 67(20): 10078-10086.

### DISCLOSURE OF THE INVENTION

The present invention relates to humanin and humanin-like peptides or a fragment or derivative thereof, for use in the treatment and/or prevention of bone- or cartilage disorders. We have identified that growth retardation in animals caused by e.g. glucocorticoids or chemotherapy was completely rescued by using humanin-like peptides. To date, there are no other drugs known, which could be used in combination with glucocorticoids or chemotherapy in order to prevent their negative side effects on cartilage tissues and/or bone tissues. The findings that the use of humanin or humanin-like peptides in combination with dexamethasone or chemotherapy completely prevents the negative effects of dexamethasone or chemotherapy, wihout interfering with the primary effects of the drugs is of considerable clinical importance.

Thus, in one aspect of the invention there is provided humanin or a humanin-like peptide (HLP) or a fragment, analog or derivative thereof, for use in the treatment and/or prevention of a disorder that negatively affects cartilage tissues and/or bone tissues.

In another aspect of the invention there is provided a humanin or a humanin-like peptide (HLP) or a fragment, analog or derivative thereof, for use in the treatment and/or prevention of a bone- or cartilage disorder.

In one embodiment of this aspect, said disorder is drug-induced.

In another embodiment of this aspect, said peptide comprises an amino acid sequence selected from SEQ ID NO:1 to SEQ ID NO:21, as well as an amino acid sequence having at least 60%, at least 65%, at least 70%, at least 75%, at least 80%, or at least 85% sequence identity to the sequences defined in SEQ ID NO:1 to SEQ ID NO:21.

In another embodiment of this aspect, said peptide comprises an amino acid sequence selected from SEQ ID NO:1 to SEQ ID NO:21.

In another embodiment of this aspect, said peptide comprises comprising the amino acid sequence of SEQ ID NO:2.

In another embodiment of this aspect, said bone- or cartilage disorder is either a primary or secondary bone- or cartilage disorder.

By a disorder is meant a disease or condition leading to an abnormality of normal functions. Such a condition includes, in accordance with the present invention, articular cartilage damage which if untreated can lead to osteoarthritis or osteoarthrosis. Another such a condition is delayed fracture healing which if untreated can lead to severe consequences including pseudoarthrosis.

By a primary bone or cartilage disorder is meant growth impairment, short stature, arthritis, osteoarthritis, rheumatoid arthritis, psoriasis arthritis, ankylosing spondylitis, osteoarthrosis, osteomyelitis fibrous dysplasia, fibrodysplasia ossificans, craniosynostosis, metabolic bone disease, osteitis deformans, osteogenesis imperfecta, scoliosis, leg length difference, bone dysplasia, osteopetrosis, osteomalacia, osteopenia, osteoporosis, osteitis fibrosa cystic, osteochondritis, osteonecrosis, osteosarcoma, bone tumor, osteochondroma, osteochondropathy, chondropathy, chondrodysplasia, achondrodysplasia, hypochondrodysplasia, chondrodystrophy, chondromalacia patellae, articular cartilage damage and delayed fracture healing.

By a secondary bone or cartilage disorder is meant a disorder which is a consequence of another disorder not primarily involving bone or cartilage tissues. Such another disorder includes Cushing's syndrome which is caused by increased cortisol production.

By undesired immune response or undesired inflammatory response is meant responses which have undesired effects on bone or cartilage tissues.

In another embodiment of this aspect, said disorder is drug-induced from drugs used in the treatment of rheumatological disorders, respiratory disorders, gastrointestinal disorders, cardiovascular disorders, endocrinological disorders, cancer, neurodegenerative disorders, kidney disorders, liver disorders, dermatologic disorders, allergic disorders, inflammatory disorders, metabolic disorders, undesired immune response, undesired inflammatory response, obesity or diabetes.

Rheumatological disorders include, but are not limited to, reumatoid arhtritis, systemic lupus erythematosus, vasculitis, periarhteritis nodosa and sarcoidosis. Respiratory disorders include, but are not limited to, asthma bronchiale, chronic obstructive lung disease and bronchiolitis obliterans. Gastrointestinal disorders include, but are not limited to, colitis ulcerosa, Crohn's disease and pancreatitis. Cardiovascular disorders include, but are not limited to, myocarditis, ischemia, hemolytic anemia and granulocytopenia. Endocrinological disorders include, but are not limited to, adrenal disorder, glucocorticoid deficiency, congenital adrenal hyperplasia, adrenoleukodystrophy and pituitary disorder. Cancer includes, but is not limited to, acute myeloid leukemia, multiple myeloma and lymphoma. Neurodegenerative disorders include, but are not limited to, multiple sclerosis and encephalitis. Kidney disorders include, but are not limited to, glomerulonephritis and nephrotic syndrome. Liver disorders include, but are not limited to, hepatitis and cholangitis. Dermatologic disorders include, but are not limited to, psoriasis, exzema, myositis, dermatomyositis and dermatitis.

In another embodiment of this aspect, said peptide is for use in the treatment and/or prevention of drug-induced bone growth impairment, short stature and osteoporosis.

In another embodiment of this aspect, said peptide improves bone growth.

In another embodiment of this aspect, said peptide prevents development of osteoporosis.

In another embodiment of this aspect, said disorder is drug-induced from an anti-inflammatory drug, preferably a glucocorticoid drug. Said glucocorticoid drug may be selected from hydrocortisone, hydrocortisone buteprate, hydrocortisone butyrate, budesonide, ciclesonide, cortisone acetate, deflazacort, medrysone, tixocortol, cloprednol, halcinonide, pregnadiene, rimexolone, flunisolide, triamcinolone, amcinonide, fluocinolone acetonide, fluocinonide, fluorometholone, clocortolone, diflucortolone, fluocortin, desoximetasone, prednisone, prednisolone, methylprednisolone, methylprednisolone aceponate, prednicarbate, prednylidene, desonide, fluprednisolone, difluprednate, fluperolone, meprednisone, dexamethasone, betamethasone, triamcinolone, beclometasone, clobetasone, diflorasone, halometasone, ulobetasol, fludrocortisone acetate, beclomethasone dipropionate, beclomethasone monopropionate, paramethasone, alclometasone, fluclorolone, flumetasone, fluprednidene, triamcinolone, flunisolide, cortivazol, fluticasone, fluticasone propionate, fluticasone furoate, loteprednol, fludroxycortide, formocortal and mometasone furoate. Preferably, said glucocorticoid drug is dexamethasone.

In another embodiment of this aspect, said disorder is drug-induced from an anti-cancer drug, prefarably a proteasome inhibitor, such as bortezomib, MLN9708, MLN273, MLN519, NPI-0052, disulfiram, ritonavir, lactacystin, CEP-18770, carfilzomib, PS-519, MG132, MG 262, ALLN, fellutamide B, tyropeptin A, omuralide, salinosporamide A, eponeomycine, epoxomicin, TMC-95A, syringolin A, glidobactin A, TMC-95 analogs, NLVS and ZLVS. Preferably, said proteasome inhibitor is bortezomib.

In another embodiment of this aspect, said disorder is drug-induced from a GnRH agonist drug, such as leuprolide, buserelin, nafarelin, histrelin, goserelin and deslorelin.

In another embodiment of this aspect, said disorder is drug-induced from a GnRH antagonist drug, such as cetrorelix, ganirelix, abarelix and degarelix.

In another embodiment of this aspect, said disorder is drug-induced from a selective estrogen receptor modulator drug, such as tamoxifen, afimoxifene, 4-hydroxytamoxifen, arzoxifene, bazedoxifene, clomifene, femarelle, lasofoxifene, ormeloxifene, raloxifene and tamoxifentoremifene.

In another embodiment of this aspect, said disorder is drug-induced from an anti-androgen drug, such as spironolactone, cyproterone acetate, flutamide, nilutamide, bicalutamide, ketoconazole, finasteride and dutasteride.

In another embodiment of this aspect, said disorder is drug-induced from an aromatase inhibitor drug, such as letrozole, anastrozole, exemestane, vorozole, formestane, fadrozole, aminoglutethimide and testolactone.

In another embodiment of this aspect, said peptide is administered in combination with another drug known to induce a bone- or cartilage disorder. Said another drug may be selected from an anti-inflammatory drug, a selective estrogen receptor modulator drug, an anti-androgen drug, an aromatase inhibitor drug and an anti-cancer drug, preferably an anti-cancer drug or an anti-inflammatory drug. Such anti-inflammatory drug may be a glucocorticoid drug, such as hydrocortisone, hydrocortisone buteprate, hydrocortisone butyrate, budesonide, ciclesonide, cortisone acetate, deflazacort, medrysone, tixocortol, cloprednol, halcinonide, pregnadiene, rimexolone, flunisolide, triamcinolone, amcinonide, fluocinolone acetonide, fluocinonide, fluorometholone, clocortolone, diflucortolone, fluocortin, desoximetasone, prednisone, prednisolone, methylprednisolone, methylprednisolone aceponate, prednicarbate, prednylidene, desonide, fluprednisolone, difluprednate, fluperolone, meprednisone, dexamethasone, betamethasone, triamcinolone, beclometasone, clobetasone, diflorasone, halometasone, ulobetasol, fludrocortisone acetate, beclomethasone dipropionate, beclomethasone monopropionate, paramethasone, alclometasone, fluclorolone, flumetasone, fluprednidene, triamcinolone, flunisolide, cortivazol, fluticasone, fluticasone propionate, fluticasone furoate, loteprednol, fludroxycortide, formocortal and mometasone furoate, preferably dexamethasone.

Such an anti-cancer drug may be a proteasome inhibitor, such as bortezomib, MLN9708, MLN273, MLN519, NPI-0052, disulfiram, ritonavir, lactacystin,

CEP-18770, carfilzomib, PS-519, MG132, MG 262, ALLN, fellutamide B, tyropeptin A, omuralide, salinosporamide A, eponeomycine, epoxomicin, TMC-95A, syringolin A, glidobactin A, TMC-95 analogs, NLVS and ZLVS, preferably bortezomib.

In another embodiment of this aspect, said peptide is administered in combination with another drug known to induce a bone- or cartilage disorder, such as leuprolide, buserelin, nafarelin, histrelin, goserelin, deslorelin, cetrorelix, ganirelix, abarelix, degarelix, letrozole, anastrozole, exemestane, vorozole, formestane, fadrozole, aminoglutethimide, testolactone, tamoxifen, afimoxifene, 4-hydroxytamoxifen, arzoxifene, bazedoxifene, clomifene, femarelle, lasofoxifene, ormeloxifene, raloxifene, tamoxifentoremifene, spironolactone, cyproterone acetate, flutamide, nilutamide, bicalutamide, ketoconazole, finasteride, dutasteride, cyclophosphamide, mechlorethamine, uramustine, melphalan, chlorambucil, ifosfamide, carmustine, lomustine, streptozocin, busulfan, thiotepa, cisplatin, carboplatin, nedaplatin, oxaliplatin, satraplatin, triplatin tetranitrate, procarbazine, altretamine, dacarbazine, mitozolomide, temozolomide, imatinib mesylate, erlotinib, gefitinib, sunitinib, roscovitine, bevacizumab, rapamycin, cyclosporin A, tacrolimus, 5-fluorouracil, methotrexate, etoposide, doxorubicin, actinomycine, vitamin A acid, 13-cis-retinoic acid, 2-chlorodeoxyadenosine, 5-azacitidine, 5-fluorouracil, 6-mercaptopurine, 6-thioguanine, abraxane, accutane, actinomycin-d, adriamycin, adrucil, afinitor, agrylin, ala-cort, aldesleukin, alemtuzumab, alitretinoin, alkaban-aq, alkeran, all-transretinoic acid, alpha interferon, altretamine, amethopterin, amifostine, aminoglutethimide, anagrelide, anandron, anastrozole, arabinosylcytosine, ara-c, aranesp, aredia, arimidex, aromasin, arranon, arsenic trioxide, asparaginase, avastin, azacitidine, bendamustine, bevacizumab, bexarotene, bicalutamide, blenoxane, bleomycin, busulfan, busulfex, calcium leucovorin, campath, camptosar, camptothecin-11, capecitabine, carac, carboplatin, carmustine, carmustine wafer, casodex, cerubidine, cetuximab, chlorambucil, cisplatin, citrovorum factor, cladribine, cortisone, cosmegen, cyclophosphamide, cytadren, cytarabine, cytarabine liposomal, cytosar-U, cytoxan, dacarbazine, dacogen, dactinomycin, darbepoetin alfa, dasatinib, daunomycin, daunorubicin, daunorubicin hydrochloride, daunorubicin liposomal, daunoxome, decadron, decitabine, delta-cortef, deltasone, denileukin diftitox, depocyt, dexamethasone acetate, dexamethasone sodium phosphate, dexasone, dexrazoxane, dhad, dic, diodex, docetaxel, doxil, doxorubicin, doxorubicin liposomal, droxia, duralone, efudex, eligard, ellence, eloxatin, elspar, emcyt, epirubicin, epoetin alfa, erbitux, erlotinib, erwinia L-asparaginase, estramustine, ethyol, etopophos, etoposide, etoposide phosphate, eulexin, everolimus, evista, exemestane, fareston, faslodex, femara, filgrastim, floxuridine, fludara, fludarabine, fluoroplex, fluorouracil, fluorouracil (cream), fluoxymesterone, flutamide, folinic acid, fulvestrant, gefitinib, gemcitabine, gemtuzumab ozogamicin, gemzar, gleevec, gliadel wafer, goserelin, halotestin, herceptin, hexadrol, hexalen, hexamethylmelamine, hycamtin, hydrea, hydrocort acetate, hydrocortisone, hydrocortisone sodium phosphate, hydrocortisone sodium succinate, hydrocortone phosphate, hydroxyurea, ibritumomab, ibritumomab tiuxetan, idamycin, idarubicin, ifex, ifosfamide, imatinib mesylate, imidazole carboxamide, interferon alfa, interleukin-2, interleukin-11, intron A (interferon alfa-2b), iressa, irinotecan, isotretinoin, ixabepilone, ixempra, kidrolase (t), lanacort, lapatinib, l-asparaginase, lenalidomide, letrozole, leucovorin, leukeran, leukine, leuprolide, leurocristine, leustatin, liposomal ara-C, liquid pred, lomustine, L-sarcolysin, lupron, lupron depot, matulane, maxidex, mechlorethamine, mechlorethamine hydrochloride, medralone, medrol, megace, megestrol, megestrol acetate, melphalan, mercaptopurine, mesna, mesnex, methotrexate, methotrexate sodium, methylprednisolone, meticorten, mitomycin, mitomycin-c, mitoxantrone, m-prednisol, mustargen, mustine, mutamycin, myleran, mylocel, mylotarg, navelbine, nelarabine, neosar, neulasta, neumega, neupogen, nexavar, nilandron, nilutamide, nipent, nitrogen mustard, novaldex, novantrone, nplate, octreotide, octreotide acetate, oncospar, oncovin, ontak, onxal, oprelvekin, orapred, orasone, oxaliplatin, paclitaxel, paclitaxel protein-bound, pamidronate, panitumumab, panretin, paraplatin, pediapred, PEG Interferon, pegaspargase, pegfilgrastim, peginterferon alfa-2b, PEG-L-asparaginase, pemetrexed, pentostatin, phenylalanine mustard, platinol, platinol-AQ, prednisolone, prednisone, prelone, procarbazine, proleukin, prolifeprospan 20 with carmustine implant, purinethol, raloxifene, revlimid, rheumatrex, rituxan, rituximab, romiplostim, rubex, rubidomycin hydrochloride, sandostatin, sandostatin LAR, sargramostim, solu-cortef, solu-medrol, sorafenib, streptozocin, sunitinib, sutent, tamoxifen, tarceva, targretin, taxol, taxotere, temodar, temozolomide, temsirolimus, teniposide, thalidomide, thalomid, theracys, thioguanine, thioguanine tabloid, thiophosphoamide, thioplex, thiotepa, toposar, topotecan, toremifene, torisel, tositumomab, trastuzumab, treanda, tretinoin, trexall, trisenox, vectibix, velban, vepesid, vesanoid, viadur, vidaza, vinblastine, vinblastine sulfate, vincasar pfs, vincristine, vinorelbine, vinorelbine tartrate, vorinostat, vumon, xeloda, zanosar, zevalin, zinecard, zoladex, zoledronic acid, zolinza and zometa.

In another aspect of the invention there is provided humanin or a humanin-like peptide (HLP) or a fragment, analog or derivative thereof, for use in the treatment and/or prevention of a disorder that negatively affects cartilage tissues and/or bone tissues, wherein said disorder that negatively affects cartilage tissues and/or bone tissues is selected from short stature, bone dysplasia, osteoporosis, osteomalacia, cancer, endocrinological disease, rheumatological disease, inflammation, infectious disease, diabetes, obesity, metabolic disease, gastrointestinal disease, neurodegenerative disease, autoimmune disease and cardiovascular disease.

In another aspect of the invention there is provided humanin or a humanin-like peptide (HLP) or a fragment, analog or derivative thereof, for use in the treatment and/or prevention of a disorder that negatively affects cartilage tissues and/or bone tissues, wherein said disorder that negatively affects cartilage tissues and/or bone tissues is selected from short stature, osteoporosis and cancer.

In another aspect of the invention, there is provided a method for treating and/or preventing a bone- or cartilage disorder, comprising administering to a patient in need of treatment, an effective amount of humanin or a humanin-like peptide (HLP) or a fragment, analog or derivative thereof.

In one embodiment of this aspect, said bone- or cartilage disorder represents a drug-induced bone- or cartilage disorder.

In another embodiment of this aspect, said peptide is selected from an amino acid sequence selected from SEQ ID NO:1 to SEQ ID NO:21, as well as an amino acid sequence having at least 60%, at least 65%, at least 70%, at least 75%, at least 80%, or at least 85% sequence identity to the peptide sequences defined in SEQ ID NO:1 to SEQ ID NO:21.

In another embodiment of this aspect, said comprises an amino acid sequence selected from SEQ ID NO:1 to SEQ ID NO:21.

In another embodiment of this aspect, said peptide comprises an amino acid sequence of SEQ ID NO:2.

In another embodiment of this aspect, said bone- or cartilage disorder is either primary or secondary bone- or cartilage disorder.

In another embodiment of this aspect, said drug-induced bone or cartilage disorder is drug-induced by drugs used in the treatment of rheumatological disorders, respiratory disorders, gastrointestinal disorders, cardiovascular disorders, endocrinological disorders, cancer, neurodegenerative disorders, kidney disorders, liver disorders, dermatologic disorders, allergic disorders, inflammatory disorders, metabolic disorders, undesired immune response, undesired inflammatory response, obesity or diabetes.

In another embodiment of this aspect, said drug-induced bone or cartilage disorder is selected from bone growth impairment, short stature and osteoporosis.

In another embodiment of this aspect, said peptide improves bone growth.

In another embodiment of this aspect, said peptide prevents development of osteoporosis.

In another embodiment of this aspect, said drug-induced bone or cartilage disorder is induced by an anti-inflammatory drug, such as a glucocorticoid drug. Said glucocorticoid drug may be selected from hydrocortisone, hydrocortisone buteprate, hydrocortisone butyrate, budesonide, ciclesonide, cortisone acetate, deflazacort, medrysone, tixocortol, cloprednol, halcinonide, pregnadiene, rimexolone, flunisolide, triamcinolone, amcinonide, fluocinolone acetonide, fluocinonide, fluorometholone, clocortolone, diflucortolone, fluocortin, desoximetasone, prednisone, prednisolone, methylprednisolone, methylprednisolone aceponate, prednicarbate, prednylidene, desonide, fluprednisolone, difluprednate, fluperolone, meprednisone, dexamethasone, betamethasone, briamcinolone, beclometasone, clobetasone, diflorasone, halometasone, ulobetasol, fludrocortisone acetate, beclomethasone dipropionate, beclomethasone monopropionate, paramethasone, alclometasone, fluclorolone, flumetasone, fluprednidene, triamcinolone, flunisolide, cortivazol, fluticasone, fluticasone propionate, fluticasone furoate, loteprednol, fludroxycortide, formocortal and mometasone furoate, preferably dexamethasone.

In another embodiment of this aspect, said drug-induced bone or cartilage disorder is induced by an anti-cancer drug, such as a proteasome inhibitor. Said proteasome inhibitor may be selected from bortezomib, MLN9708, MLN273, MLN519, NPI-0052, disulfiram, ritonavir, lactacystin, CEP-18770, carfilzomib, PS-519, MG132, MG 262, ALLN, fellutamide B, tyropeptin A, omuralide, salinosporamide A, eponeomycine, epoxomicin, TMC-95A, syringolin A, glidobactin A, TMC-95 analogs, NLVS and ZLVS, preferably bortezomib.

In another embodiment of this aspect, said drug-induced bone or cartilage disorder is induced by a GnRH agonist drug, such as leuprolide, buserelin, nafarelin, histrelin, goserelin and deslorelin.

In another embodiment of this aspect, said drug-induced bone or cartilage disorder is induced by a GnRH antagonist drug, such as cetrorelix, ganirelix, abarelix and degarelix.

In another embodiment of this aspect, said drug-induced bone or cartilage disorder is induced by a selective estrogen receptor modulator drug, such as tamoxifen, afimoxifene, 4-hydroxytamoxifen, arzoxifene, bazedoxifene, clomifene, femarelle, lasofoxifene, ormeloxifene, raloxifene and tamoxifentoremifene.

In another embodiment of this aspect, said drug-induced bone or cartilage disorder is induced by an anti-androgen drug, such as spironolactone, cyproterone acetate, flutamide, nilutamide, bicalutamide, ketoconazole, finasteride and dutasteride.

In another embodiment of this aspect, said drug-induced bone or cartilage disorder is induced by an aromatase inhibitor drug, such as letrozole, anastrozole, exemestane, vorozole, formestane, fadrozole, aminoglutethimide, and testolactone.

In another embodiment of this aspect, said peptide is administered in combination with a drug known to induce a bone- or cartilage disorder. Said drug may be selected from an anti-inflammatory drug, a selective estrogen receptor modulator drug, an anti-androgen drug, an aromatase inhibitor drug and an anti-cancer drug, preferably an anti-inflammatory drug or an anti-cancer drug. Said anti-inflammatory drug, may be a glucocorticoid drug, such as hydrocortisone, hydrocortisone buteprate, hydrocortisone butyrate, budesonide, ciclesonide, cortisone acetate, deflazacort, medrysone, tixocortol, cloprednol, halcinonide, pregnadiene, rimexolone, flunisolide, triamcinolone, amcinonide, fluocinolone acetonide, fluocinonide, fluorometholone, clocortolone, diflucortolone, fluocortin, desoximetasone, prednisone, prednisolone, methylprednisolone, methylprednisolone aceponate, prednicarbate, prednylidene, desonide, fluprednisolone, difluprednate, fluperolone, meprednisone, dexamethasone, betamethasone, briamcinolone, beclometasone, clobetasone, diflorasone, halometasone, ulobetasol, fludrocortisone acetate, beclomethasone dipropionate, beclomethasone monopropionate, paramethasone, alclometasone, fluclorolone, flumetasone, fluprednidene, triamcinolone, flunisolide, cortivazol, fluticasone, fluticasone propionate, fluticasone furoate, loteprednol, fludroxycortide, formocortal and mometasone furoate. Preferably, said glucocorticoid drug is dexamethasone.

Said anti-cancer drug, may be a proteasome inhibitor, such as bortezomib, MLN9708, MLN273, MLN519, NPI-0052, disulfiram, ritonavir, lactacystin, CEP-18770, carfilzomib, PS-519, MG132, MG 262, ALLN, fellutamide B, tyropeptin A, omuralide, salinosporamide A, eponeomycine, epoxomicin, TMC-95A, syringolin A, glidobactin A, TMC-95 analogs, NLVS or ZLVS. Preferably, said proteasome inhibitor is bortezomib.

In another embodiment of this aspect, said peptide is administered in combination with a drug known to induce a bone- or cartilage disorder, said drug being selected from leuprolide, buserelin, nafarelin, histrelin, goserelin, deslorelin, cetrorelix, ganirelix, abarelix, degarelix, letrozole, anastrozole, exemestane, vorozole, formestane, fadrozole, aminoglutethimide, testolactone, tamoxifen, afimoxifene, 4-hydroxytamoxifen, arzoxifene, bazedoxifene, clomifene, femarelle, lasofoxifene, ormeloxifene, raloxifene, tamoxifentoremifene, spironolactone, cyproterone acetate, flutamide, nilutamide, bicalutamide, ketoconazole, finasteride, dutasteride, cyclophosphamide, mechlorethamine, uramustine, melphalan, chlorambucil, ifosfamide, carmustine, lomustine, streptozocin, busulfan, thiotepa, cisplatin, carboplatin, nedaplatin, oxaliplatin, satraplatin, triplatin tetranitrate, procarbazine, altretamine, dacarbazine, mitozolomide, temozolomide, imatinib mesylate, erlotinib, gefitinib, sunitinib, roscovitine, bevacizumab, rapamycin, cyclosporin A, tacrolimus, 5-fluorouracil, methotrexate, etoposide, doxorubicin, actinomycine, vitamin A acid, 13-cis-retinoic acid, 2-chlorodeoxyadenosine, 5-azacitidine, 5-fluorouracil, 6-mercaptopurine, 6-thioguanine, abraxane, accutane, actinomycin-d, adriamycin, adrucil, afinitor, agrylin, ala-cort, aldesleukin, alemtuzumab, alitretinoin, alkaban-aq, alkeran, all-transretinoic acid, alpha interferon, altretamine, amethopterin, amifostine, aminoglutethimide, anagrelide, anandron, anastrozole, arabinosylcytosine, ara-c, aranesp, aredia, arimidex, aromasin, arranon, arsenic trioxide, asparaginase, avastin, azacitidine, bendamustine, bevacizumab, bexarotene, bicalutamide, blenoxane, bleomycin, busulfan, busulfex, calcium leucovorin, campath, camptosar, camptothecin-11, capecitabine, carac, carboplatin, carmustine, carmustine wafer, casodex, cerubidine, cetuximab, chlorambucil, cisplatin, citrovorum factor, cladribine, cortisone, cosmegen, cyclophosphamide, cytadren, cytarabine, cytarabine liposomal, cytosar-U, cytoxan, dacarbazine, dacogen, dactinomycin, darbepoetin alfa, dasatinib, daunomycin, daunorubicin, daunorubicin hydrochloride, daunorubicin liposomal, daunoxome, decadron, decitabine, delta-cortef, deltasone, denileukin diftitox, depocyt, dexamethasone acetate, dexamethasone sodium phosphate, dexasone, dexrazoxane, dhad, dic, diodex, docetaxel, doxil, doxorubicin, doxorubicin liposomal, droxia, duralone, efudex, eligard, ellence, eloxatin, elspar, emcyt, epirubicin, epoetin alfa, erbitux, erlotinib, erwinia L-asparaginase, estramustine, ethyol, etopophos, etoposide, etoposide phosphate, eulexin, everolimus, evista, exemestane, fareston, faslodex, femara, filgrastim, floxuridine, fludara, fludarabine, fluoroplex, fluorouracil, fluorouracil (cream), fluoxymesterone, flutamide, folinic acid, fulvestrant, gefitinib, gemcitabine, gemtuzumab ozogamicin, gemzar, gleevec, gliadel wafer, goserelin, halotestin, herceptin, hexadrol, hexalen, hexamethylmelamine, hycamtin, hydrea, hydrocort acetate, hydrocortisone, hydrocortisone sodium phosphate, hydrocortisone sodium succinate, hydrocortone phosphate, hydroxyurea, ibritumomab, ibritumomab tiuxetan, idamycin, idarubicin, ifex, ifosfamide, imatinib mesylate, imidazole carboxamide, interferon alfa, interleukin-2, interleukin-11, intron A (interferon alfa-2b), iressa, irinotecan, isotretinoin, ixabepilone, ixempra, kidrolase (t), lanacort, lapatinib, 1-asparaginase, lenalidomide, letrozole, leucovorin, leukeran, leukine, leuprolide, leurocristine, leustatin, liposomal ara-C, liquid pred, lomustine, L-sarcolysin, lupron, lupron depot, matulane, maxidex, mechlorethamine, mechlorethamine hydrochloride, medralone, medrol, megace, megestrol, megestrol acetate, melphalan, mercaptopurine, mesna, mesnex, methotrexate, methotrexate sodium, methylprednisolone, meticorten, mitomycin, mitomycin-c, mitoxantrone, m-prednisol, mustargen, mustine, mutamycin, myleran, mylocel, mylotarg, navelbine, nelarabine, neosar, neulasta, neumega, neupogen, nexavar, nilandron, nilutamide, nipent, nitrogen mustard, novaldex, novantrone, nplate, octreotide, octreotide acetate, oncospar, oncovin, ontak, onxal, oprelvekin, orapred, orasone, oxaliplatin, paclitaxel, paclitaxel protein-bound, pamidronate, panitumumab, panretin, paraplatin, pediapred, PEG Interferon, pegaspargase, pegfilgrastim, peginterferon alfa-2b, PEG-L-asparaginase, pemetrexed, pentostatin, phenylalanine mustard, platinol, platinol-AQ, prednisolone, prednisone, prelone, procarbazine, proleukin, prolifeprospan 20 with carmustine implant, purinethol, raloxifene, revlimid, rheumatrex, rituxan, rituximab, romiplostim, rubex, rubidomycin hydrochloride, sandostatin, sandostatin LAR, sargramostim, solu-cortef, solu-medrol, sorafenib, streptozocin, sunitinib, sutent, tamoxifen, tarceva, targretin, taxol, taxotere, temodar, temozolomide, temsirolimus, teniposide, thalidomide, thalomid, theracys, thioguanine, thioguanine tabloid, thiophosphoamide, thioplex, thiotepa, toposar, topotecan, toremifene, torisel, tositumomab, trastuzumab, treanda, tretinoin, trexall, trisenox, vectibix, velban, vepesid, vesanoid, viadur, vidaza, vinblastine, vinblastine sulfate, vincasar pfs, vincristine, vinorelbine, vinorelbine tartrate, vorinostat, vumon, xeloda, zanosar, zevalin, zinecard, zoladex, zoledronic acid, zolinza and zometa.

In another aspect of the invention, there is provided a pharmaceutical composition, comprising humanin or a humanin-like peptide (HLP) or a fragment, analog or derivative thereof, for use in the treatment and/or prevention of a disorder that negatively affects cartilage tissues and/or bone tissues, together with at least one pharmaceutically acceptable carrier or excipient.

In another aspect of the invention, there is provided a A method of treating or preventing a bone- or cartilage disorder, comprising administering to a patient in need of treatment, an effective amount of an agent that regulates/affects the local production and/or expression of humanin or another peptide expressed by the humanin gene in cartilage tissues or bone.

The humanin or a humanin-like peptide (HLP) or a fragment, analog or derivative thereof, of the invention includes, but are not limited to, the below listed peptides. The peptides are also disclosed in FIG. 10.

(SEQ ID NO: 1)

| | |
|---|---|
| Humanin (HN): | MAPRGFSCLLLLTSEIDLPVKRRA; |

(SEQ ID NO: 2)

| | |
|---|---|
| S14G-HN (HNG): | MAPRGFSCLLLLTGEIDLPVKRRA; |

(SEQ ID NO: 3)

| | |
|---|---|
| D-Ser14 HN: | MAPRGFSCLLLLT(DS)EIDLPVKRRA; |

(SEQ ID NO: 4)

| | |
|---|---|
| AGA-HNG: | MAPAGASCLLLLTGEIDLPVKRRA; |

(SEQ ID NO: 5)

| | |
|---|---|
| AGA-(D-Ser14)HN: | MAPAGASCLLLLT(DS)EIDLPVKRRA; |

(SEQ ID NO: 6)

| | |
|---|---|
| AGA-(D-Ser14)HN17: | PAGASCLLLLT(DS)EIDLP; |

(SEQ ID NO: 7)

| | |
|---|---|
| AGA-(C8R)HNG17: | PAGASRLLLLTGEIDLP; |

(SEQ ID NO: 8)

| | |
|---|---|
| EF-HN: | EFLIVIKSMAPRGFSCLLLLTSEIDLPVKRRA; |

(SEQ ID NO: 9)

| | |
|---|---|
| EF-HNG: | EFLIVIKSMAPRGFSCLLLLTGEIDLPVKRRA; |

(SEQ ID NO: 10)

| | |
|---|---|
| EF-AGA-HNG: | EFLIVIKSMAPAGASCLLLLTGEIDLPVKRRA; |

(SEQ ID NO: 11)

| | |
|---|---|
| Colivelin: | SALLRSIPAPAGASRLLLLTGEIDLP; |

(SEQ ID NO: 12)

| | |
|---|---|
| L9R-HN: | MAPRGFSCRLLLTSEIDLPVKRRA; |

(SEQ ID NO: 13)

| | |
|---|---|
| Humanin (7): | MTPRGFSCLLLPTSETDLPVKRRX; |

(SEQ ID NO: 14)

| | |
|---|---|
| Humanin (5): | MAPRGFSCLLLSTSEIDLPVKRXX; |

(SEQ ID NO: 15)

| | |
|---|---|
| Humanin (3/11): | MAPRGFSCLLLSTSEIDLPVKRRA; |

(SEQ ID NO: 16)

| | |
|---|---|
| SHLP1: | MCHWAGGASNTGDARGDVFGKQAG; |

(SEQ ID NO: 17)

| | |
|---|---|
| SHLP2: | MGVKFFTLSTRFFPSVQRAVPLWTNS; |

(SEQ ID NO: 18)

| | |
|---|---|
| SHLP3: | MLGYNFSSFPCGTISIAPGFNFYRLYFIWVNGL |
| | AKVVW; |

(SEQ ID NO: 19)

| | |
|---|---|
| SHLP4: | MLEVMFLVNRRGKICRVPFTFFNLSL; |

(SEQ ID NO: 20)

| | |
|---|---|
| SHLP5: | MYCSEVGFCSEVAPTEIFNAGLVV; |
| and | |

(SEQ ID NO: 21)

| | |
|---|---|
| SHLP6: | MLDQDIPMVQPLLKVRLFND. |

Each X indicated in SEQ ID NO:13 and SEQ ID NO:14 optionally represent any amino acid.

Further humanin-like peptides (HLP) are also part of the invention, including peptides disclosed in WO 2009/135165; Table 3, pages 9-11 and Table 5, pages 14-22.

Also provided herein are variants, comprising an amino acid sequence having at least 60%, at least 65%, at least 70%, at least 75%, at least 80%, or at least 85% sequence identity to the amino acid sequence of Table 1, in FIG. 10.

In some aspects, the peptides of the invention have therapeutic activity of exerting its beneficial effects in a wide range of cartilage and bone disorders.

In some aspects, the peptides of the invention have a therapeutic activity of exerting its beneficial effects on cartilage tissues and/or bone tissues by themselves when given alone.

In some aspects, the peptides of the invention have a therapeutic activity of exerting its beneficial effects on cartilage disorders and/or bone disorders such as decreased bone growth and/or osteoporosis caused by different drugs, for example, but not limited to glucocorticoids and/or chemotherapeutics.

### BRIEF DESCRIPTION OF THE DRAWINGS

FIG. 1. Human bone and growth plate histology (1A) and expression/localization of humanin, in human growth plate cartilage (1B; see Example 1 for further explanation).

FIG. 2A. Growth promoting/protective effects of humanin analog HNG (S14G) on longitudinal bone growth in cultured fetal rat metatarsal bones (see Example 2 for further explanation).

FIG. 2B. Growth promoting/protective effects of humanin derivative colivelin on longitudinal bone growth in cultured fetal rat metatarsal bones (see Example 2 for further explanation).

FIG. 2C. Humanin analog HNG (S14G) does not alter the anti-inflammatory effects of Dexa, when given in combination (see Example 3 for further explanation).

FIGS. 3A and B. Humanin analog HNG (S14G) rescues mice from dexamethasone-induced growth retardation (see Example 4 for further explanation).

FIG. 3C. Humanin analog HNG (S14G) increases the bone mineral density (BMD) by its own in mice (see Example 5 for further explanation).

FIG. 3D. Humanin analog HNG (S14G) stimulates chondrogensis by regulating resting and proliferating chondrocyte zone (R+P) (see Example 6 for further explanation).

FIG. 3E. Humanin analog HNG (S14G) positively regulates chondrocyte differentiation process by regulating type-X collagen in the mouse growth plate (see Example 7 for further explanation).

FIG. 4. Growth retardation (A) and apoptosis (B) caused by CT (bortezomib). Figures showing growth of fetal (embryonic day 20) rat metatarsal bones cultured and treated in vitro with different concentrations of CT (bortezomib) (see Example 8 for further explanation).

FIG. 5. Schematic representation of the experimental design for the in vivo studies (see Example 9 for further explanation).

FIG. 6. Body weight measurement (A) in both strains of mice during and after treatment. CT (bortezomib) impairs longitudinal bone growth in vivo of two normal mouse strains (B) (see Example 9 for further explanation).

FIG. 7. CT (bortezomib) induce chondrocyte apoptosis in vivo in both strains of mice (see Example 10 for further explanation).

FIG. 8. CT (bortezomib) induce apoptosis in cultured human growth plate cartilage biopsies (obtained from young individuals) (see Example 11 for further explanation).

FIG. 9. Humanin analog HNG (S14G) protects chondrocytes from CT (bortezomib)-induced up-regulation of pro-apoptotic proteins in vitro (see Example 12 for further explanation).

FIG. 10. Humanin analog HNG (S14G) protects fetal rat metatarsal bones from CT (bortezomib)-induced growth retardation in vitro (see Example 13 for further explanation).

FIG. 11. Humanin analog HNG (S14G) rescues mice from CT (bortezomib)-induced growth retardation. Bone growth (mm) from start of treatment until end of treatment was measured in neuroblastoma- (A) or medulloblastoma tumor-xenografts of NMRI nude mice (B) (see Example 14 for further explanation).

FIG. 12. Humanin analog HNG (S14G) does not alter the anti-cancer effects of CT (bortezomib), when given in combination (see Example 15 for further explanation).

FIG. 13. Humanin analog HNG (S14G) does not alter the neuroblastoma anti-cancer effect of CT (bortezomib), when given in combination (see Example 16 for further explanation).

FIG. 14. Table with amino acid sequences of humanin and humanin analogs. Each X indicated in SEQ ID NO:13 and SEQ ID NO:14 optionally represent any amino acid.

Compositions of the invention may be administered by means which include but are not limited to intravenous, oral, subcutaneous, intra-arterial, intramuscular, intracardial, intraspinal, intrathoracic, intraperitoneal, intraventricular, sublingual, transdermal and inhalation.

The term “amino acid” refers to naturally occurring and synthetic amino acids, as well as amino acid analogs as amino acid mimetics that function in a manner similar to the naturally occurring amino acids. Naturally occurring amino acids are those encoded by the genetic code, as well as naturally occurring amino acids that are later modified. Amino acid analogs are compounds that have the same basic chemical structure as a naturally occurring amino acid. Amino acid mimetics refers to chemical compounds that have a structure that differs from the general chemical structure of an amino acid, but that functions similar to a naturally occurring amino acid.

“Endogenous” refers to a protein, nucleic acid, lipid or other biomolecule produced or originating within the body or within cells, organs, tissues of the body of a subject.

“Exogenous” refers to a protein, nucleic acid, lipid, or other biomolecule originating outside the body of a subject.

“Cartilage tissues” refers to all tissues in the body where cartilage cells (chondrocytes) are the major cellular components. This includes but is not limited to joint cartilage, growth plate cartilage and intervertebral disc cartilage.

“Bone tissues” refers to all tissues in the body where bone cells (osteocytes, osteoblasts and osteoclasts) are important cellular components. This includes but is not limited to long bones and vertebrae

“Bone growth” refers to a series of co-ordinated actions which take place at the epiphyseal growth plate of long bones by balanced cycle of cartilage growth, formation of matrix, calcification of cartilage that acts as a scaffold for bone formation and modelling (where bone is being continuously resorbed and replaced by new bone). Bone modelling is highly active in childhood/adolescence, and enables long bones to increase in diameter, change shape and develop a marrow cavity. The bone modelling process continues throughout adult life (this process in the adults is known as remodelling) with bone resorption equally balanced by bone formation in a healthy skeleton. Bone growth disorders occur when there is disruption of the normal cellular activity of chondrocytes (growth plate/articular) and/or bone cells.

“Bone metabolism” refers to a process involving bone modeling and bone remodeling where cells produce the substances and energy needed for their survival. During bone remodeling, bone resorption by osteoclasts is followed by bone formation by osteoblasts. Bone remodeling does not result in changes of bone shape but helps in repairing of microdamage. Wherase, modeling is the formation of new bone by osteoblasts at locations different from the sites of bone resorption by osteoclasts and results in bone growth.

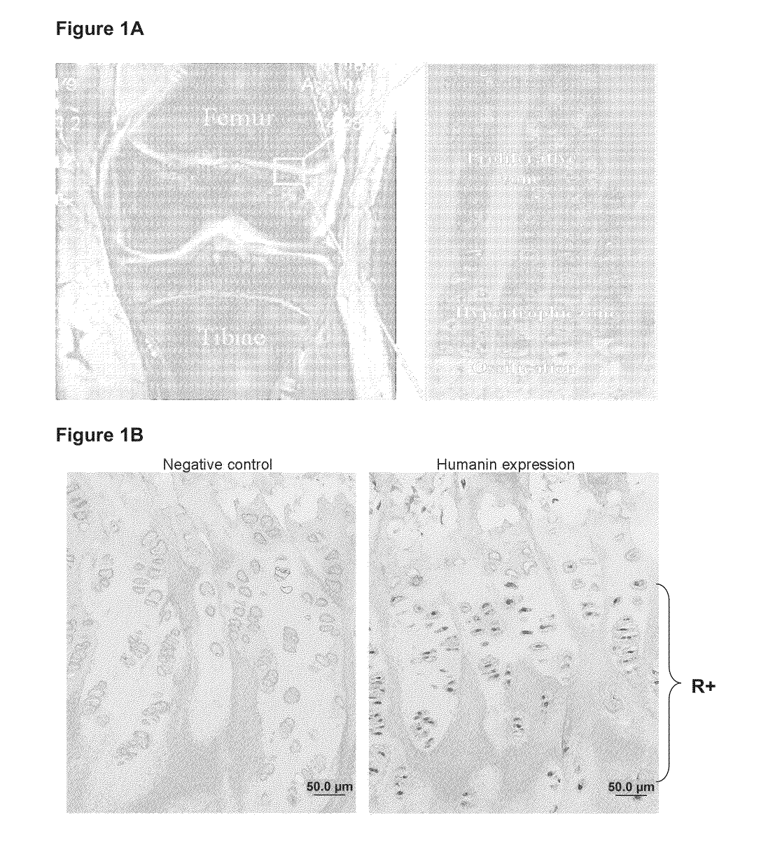

“Longitudinal bone growth” refers endochondral bone formation that occurs in the growth plates (a thin layer of cartilage located on each end of long bones, see FIG. 1A) where in a series of co-ordinated steps where resting zone/stem-like chondrocytes are recruited to start active proliferation and then undergo differentiation, followed by apoptosis and later mineralization resulting in increased bone length.

“Drug-induced longitudinal bone growth impairement” refers to a condition when drugs given to the patient/subject (mammal, children, adolescents, adults, animal) to treat different diseases but as a side effect they also alter/inhibit the normal process of bone elongation causing longitudinal bone growth impairment/retardation.

“Drug-induced” refers to an effect which is primarily due to drug(s) given to patient/subject under different disease conditions.

“Fragment” refers to a small part synthesised/produced/broken off/or detached from its original place.

“Derivative” refers to a substance derived/produced/obtained either directly or by modification or partial substitution.

“Humanin or a humanin-like peptide (HLP) or a fragment, analog or derivative thereof” refers to a polypeptide with a sequence selected from SEQ ID NO:1 to SEQ ID NO:21, as well as an amino acid sequence having at least 60%, at least 65%, at least 70%, at least 75%, at least 80%, or at least 85% sequence identity to the sequences defined in SEQ ID NO:1 to SEQ ID NO:21.

“Catch-up growth” refers to acceleration of the growth rate in infants or young children above the limits of normal for age after a transient period of growth inhibition/impairment; it can be complete or incomplete.

The term “peptide” refers to any of various natural or synthetic compounds containing at least two or more amino acids linked by the carboxyl group of one amino acid to the amino group of another.

“Polypeptide” refers to chains of amino acids, and “proteins” are made up of one or more polypeptide molecules.

“Polynucleotide” refers to a polymeric form of nucleotides of at least 10 bases or base pairs in length, either ribonucleotides or deoxynucleotides or a modified form of either type of nucleotide, including single and double stranded forms of DNA.

The terms “polypeptide”, “peptide” and “protein” herein refers to a polymer (large molecules composed of repeating structural units typically connected by covalent chemical bonds) formed from the linking, in a defined order, of α-amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “endogenous expression of humanin in cartilage tissues and/or bone tissues” refers to the local production and/or secretion of humanin within cartilage tissues and/or bone tissues.

The phrase “agent that affects the local production and/or expression of humanin or another peptide expressed by the humanin gene” refers to an agent that for example, but not limited to, triggers endogenous production of humanin or humanin like peptides/derivatives/analogs in cartilage tissues and/or bone tissues by the use of siRNA, miRNA, shRNA (plasmid and lentiviral).

“MicroRNA (miRNA)” refers to a class of small RNA molecules, about 21 nucleotides in length that regulate gene expression in a variety of ways.

“RNA interference (RNAi)” refers to a biological mechanism by which double-stranded RNA (dsRNA) induces gene silencing by targeting complementary mRNA for degradation.

“small interfering RNA (siRNA)” refers to a class of double-stranded RNA molecules, 20-25 nucleotides in length, that play a variety of roles in cell. For example, siRNA is involved in the RNA interference (RNAi) pathway, where it interferes with the expression of a specific gene.

“short hairpin RNA (shRNA)” refers to a sequence of RNA that makes a tight hairpin turn that can be used to silence gene expression via RNA interference.

“Disorder that affects the endogenous expression of humanin in cartilage tissues and/or bone tissues negatively influencing cartilage tissues and/or bone tissues” refers to the presence, absence, increased or decreased level of humanin in cartilage tissues and/or bone tissues with or without treatment causing cartilage tissue and/or bone tissue disorders. For example, a patient suffering from increased or decreased and/or insufficient levels of locally produced humanin in cartilage tissues and/or bone tissues resulted from with/without treatment of any disease.

In some aspects, humanin and humanin-like peptides and derivates, analogs and variants thereof is at least about 30%, preferably 35%, 40%, 45%, 50%, 55%, 60%, 65%, 70%, 75%, 85%, 95% or 98% identical to amino acid sequence of SEQ ID NO: 1-22.

In some aspects, humanin and humanin-like peptides (HLPs) and derivates, analogs and variants thereof has an amino acid sequence which is “substantially identical” to the sequence of the corresponding human humanin, in that the sequence is at least about 30%-98% or more identical to the sequence of a reference sequence, such as the corresponding endogenous human humanin.

A “amino acid(s) substitution” is one in which one or more amino acid reidue(s) is replaced with an amino acid residue. Families of amino acid residues have been defined in the art and include amino acids with basic side chains (e.g lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. alanine, valine, leucine, isoleucine, proline, phenylalanine, methionine, tryptphan), beta-branched side chains (e.g. threonine, valine, isoleucine) and aromatic side chains (e.g. tyrosine, phenylalanine, tryptophan, histidine). A predicted non-essential amino acid residue in humanin and humanin-like peptides (HLPs) and derivates, analogs and variants thereof can typically be replaced with another amino acid residue, preferably from the same side chain family.

A “non-essential” amino acid residue is a residue that can be altered from the original sequence (e.g. a sequence of SEQ ID NO: 1-22) without abolishing or substantially altering the therapeutic activity of the peptide, whereas an “essential” amino acid residue is a residue that cannot be altered without introducing such a change.

The terms “identical” or percent “identity”, in the context of two or more nucleic acids or polypeptide sequences, refers to two or more sequences or subsequences that are the same or have a specified percentage of amino acid residues or nucleotides that are the same when compared and aligned for maximum correspondence over a comparison window or designated region. Methods of aligning sequences for comparison are well-known in the art.

Humanin and humanin-like peptides (HLPs) and derivates, analogs and variants provided herein may be modified chemically and/or biologically. Examples of such modifications include, but are not limited to, functional group introduction such as alkylation, acylation, amidation, esterification, halogenation, amination, carboxylation, and pegylation, functional group conversion such as oxidation, reduction, addition, and elimination, glycosylation, lipid compound introduction, phosporylation, and/or biotinylation. Such modification(s) may for example stabilize and/or enhance the biological activity of humanin and humanin-like peptides (HLPs) and derivates, analogs and variants thereof.

In another aspect, identifying methods for activity of humanin comprising inhibition, and/or stimulation or expression of humanin, such as the activity or expression in the cell is modulated. Another aspect is a method for identifying modulation of transcription, splicing, translation of humanin.

Also provided herein are methods of treating a subject having, or at risk of having, a disease or condition within cartilage tissues and/or bone tissues or a disease affecting cartilage tissueas and/or bone tissues treatable by humanin or humanin-like peptides (HLPs) and derivates, analogs and variants thereof by administering an effective amount of humanin or humanin-like peptides (HLPs) and derivates, analogs and variants thereof or a pharmaceutical composition thereof to the subject. Humanin or humanin-like peptides (HLPs) and derivates, analogs and variants thereof can be administered by themselves, or they can be co-administered with one or more other agents, such as, but not limited to, hydrocortisone, cortisone acetate, prednisone, prednisolone, methylprednisolone, dexamethasone, betamethasone, triamcinolone, beclometasone, fludrocortisone acetate, beclomethasone dipropionate, beclomethasone monopropionate, triamcinolone, flunisolide, budesonide, fluticasone, a GnRH agonist (such as leuprolide, buserelin, nafarelin, histrelin, goserelin, deslorelin), a GnRH antagonist (such as cetrorelix, ganirelix, abarelix, degarelix), an Aromatase inhibitor (such as letrozole, anastrozole, exemestane, vorozole, formestane, fadrozole, aminoglutethimide, testolactone), a Selective Estrogen Receptor Modulator (such as tamoxifen, afimoxifene, 4-hydroxytamoxifen, arzoxifene, bazedoxifene, clomifene, femarelle, lasofoxifene, ormeloxifene, raloxifene, tamoxifentoremifene), an anti-androgen (eg. spironolactone, cyproterone acetate, flutamide, nilutamide, bicalutamide, ketoconazole, finasteride, dutasteride), an Alkylating agent (such as cyclophosphamide, mechlorethamine, uramustine, melphalan, chlorambucil, ifosfamide, carmustine, lomustine, streptozocin, busulfan, thiotepa, cisplatin, carboplatin, nedaplatin, oxaliplatin, satraplatin, triplatin tetranitrate, procarbazine, altretamine, dacarbazine, mitozolomide, temozolomide), a Proteasome inhibitor (such as bortezomib, NPI-0052, disulfiram, ritonavir, lactacystin, CEP-18770, carfilzomib, PS-519, MG132, MG 262, ALLN, fellutamide B, tyropeptin A, omuralide, salinosporamide A, eponeomycine, epoxomicin, TMC-95A, syringolin A, glidobactin A, TMC-95 analogs, NLVS, ZLVS), a Tyrosine kinase inhibitor (such as imatinib mesylate, erlotinib, gefitinib, sunitinib), a Cyclin-dependent kinase inhibitor (such as roscovitine), a VEGF-inhibitor (such as bevacizumab) an Immunosuppresive drug (such as rapamycin, cyclosporin A, tacrolimus), an Antimetabolite (such as 5-fluorouracil, methotrexate), etoposide, doxorubicin, actinomycine and vitamin A acid.

Humanin or humanin-like peptides (HLPs) and derivates, analogs and variants thereof can be co-administered simultaneously (in the same or separate formulations) or consecutively.

Furthermore, humanin or humanin-like peptides (HLPs) and derivates, analogs and variants thereof can be administered as an adjuvant therapy.

In some aspects, humanin or humanin-like peptides (HLPs) and derivates, analogs and variants thereof can be co-administered with one or more of a different humanin or humanin-like peptides (HLPs) and derivates, analogs and variants thereof.

The term “treating” used herein includes, but is not limited to prevention, amelioration, alleviation, and/or eliminations of disease, disorder, or condition being treated or one or more symptoms of the disease, disorder or condition being treated, but also of therapies/treatments/drugs alone or in combination affecting cartilage tissues and/or bone tissues as well as improvement in the overall well being of patient, as measured by objective and/or subjective criteria.

“Disease”, “disorder”, or “condition” herein refers to a problem affecting cartilage tissues and/or bone tissues in a negative manner, but also of therapies/treatment/drugs alone or in combination affecting cartilage tissues and or bone tissues in a negative manner.

The terms “disorder that negativly affects cartilage tissues and/or bone tissues” refers to a disorder, disease or condition that interact, alter, influences and/or disturbs the normal physiology and/or morphology and/or function of cartilage tissues and/or bone tissues. This is assessed by different means, such as, but not limited to, X-ray examination, Dual energy X-ray absorptiometry, Ultrasound, Computer tomography, Peripheral quantitative computer tomography, Magnetic resonance imaging, Visual inspection, Histological examination, Clinical examination, or Analysis of biological markers in the blood.

The term “short stature” used herein includes, but is not limited to, Familial short stature, Constitutional delay of growth and puberty, Idiopathic short stature, Small for gestational age, Intrauterine growth retardation, Growth hormone deficiency, Insulin-like growth factor-I deficiency, or growth impairment caused by a chronic disease and/or genetically determined disorder and/or syndrome.

The term “bone dysplasia” used herein includes, but is not limited to, achondrodysplasia, Hypochondrodysplasia or a disorder and/or syndrome affecting cartilage and/or bone development.

The term “osteomalacia” used herein is defined as a disorder which involves the softening of the bones due to defective bone mineralization which can be caused by, but not limited to, rickets, hypoparathyroidism, pseudohypoparathyroidism, hypophospatemia, renal tubular acidosis, cancer, malabsorption or malnutrition.

The term “osteoporosis” used herein is defined as a disease of bone that leads to an increased risk of fracture. In osteoporosis the bone mineral density is reduced and the bone microarchitecture is disrupted, and the amount and variety of proteins in bone may be altered.

A “subject” of a method provided herein refers to any mammalian patient to which peptides or compositions of the invention can be beneficially administered. The term “mammal” refers to humans and non-human primates, as well as experimental or veterinary animals, such as rabbits, rats mice, and other animals.

In some aspects, an “effective amount” of humanin or humanin-like peptides (HLPs) and derivates, analogs and variants thereof is an amount sufficient to provide a measurable reduction in symptoms or other beneficial effect(s) with respect to a disease, therapy, disorder, or condition targeted for treatment.

“Cancer” refers generally to a disease characterized by uncontrolled, abnormal cell growth and proliferation. A “tumor” or “neoplasm” is an abnormal mass of tissue that results from excessive, uncontrolled, and progressive cell division.

The present invention also provides methods of treating a subject having a disorder affecting cartilage tissues and/or bone tissues characterized by aberrant activity and/or aberrant expression of humanin or variant thereof, by administering an agent which is a modulator of the activity of humanin or variant or modulator thereof.

Also provided herein are pharmaceutical compositions comprising one or more peptides of the invention together with at least one pharmaceutically acceptable carrier or excipient.

Peptides of the invention can be incorporated into pharmaceutical compositions suitable for administration. Such compositions typically comprise the peptide and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze- drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For intranasal administration, the compounds are delivered in the form of intranasal-spray or in the form of fluid containing different excipeinets, diluents, preservatives and antioxudents (alkyl methyl sulphoxides, pyrrolidones, 1-dodecyleazacycloheptan-2-one, surfactants, parabens, benzalkonium chloride, phenyl ethyl alcohol, etc).

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives.

Transmucosal administration can be accomplished through the use of nasal sprays or suppositories. For transdermal administration, the active compounds are formulated into ointments, salves, gels, or creams as generally known in the art.

In one aspect, the active peptides are prepared with carriers that will protect the peptide against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The active ingredient of the pharmaceutical composition of the present invention may be DNA encoding the polypeptide of the present invention. When the DNA encoding the oligopeptide is used as a gene therapy agent for the disease described above, examples of administration methods thereof include a method which administers a vector incorporating the DNA therein. Examples of the vector include plasmids, adenovirus vectors, adeno-associated virus vectors, herpes virus vectors, vaccinia virus vectors, and retrovirus vectors. The therapeutic agent can be expressed in vivo with efficiency by infecting organisms with the viral vectors. Alternatively, a method which introduces the vector or the DNA into liposomes (e.g., positively charged liposomes and positively charged cholesterol) and administers the liposome can be used as an effective therapy.

When the pharmaceutical composition of the present invention is used as a preventive and/or therapeutic agent for the diseases described above, it can be administered to mammals such as humans, mice, rats, rabbits, dogs, and cats. The dose and number of doses of the pharmaceutical drug of the present invention may be changed appropriately according to the age, sex, and conditions of a subject to be administered, or administration routes.

A therapeutically effective amount of protein or polypeptide provided herein can range from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Having now generally described various aspects and aspects of the invention, the same will be more readily understood through reference to the following examples which are provided by way of illustration, and are not intended to be limiting, unless specified.

### GENERAL MATERIAL AND METHODS

### Human Growth Plate Biopsies

Tissue samples were taken from a 14 years old boy (suffering from extreme tall stature) subjected to epiphyseal surgery performed an orthopedic surgeon to arrest longitudinal leg growth. In connection with this surgery, biopsies from the tibial and femoral growth plates were obtained using a bone marrow biopsy needle (8 gauge; Gallini Medical Products and Services, Modena, Italy). The biopsies were collected in 1×PBS and immediately cut into small pieces and fixed in 4% formaldehyde for 24 hrs at 4 C. After fixation, the biopsies were decalcified in 10% EDTA for 24 hrs at 4 C and then embedded in paraffin and later on sectioned for immunohistochemical analysis.

Immunohistochemistry of Humanin

For immunohistochemistry of humanin in human growth plate chondrocytes/cartilage, the sections were deparaphinised in xylene (2×10 min), 99% ethanol (2×5 min), 95% ethanol (1×10 min), 70% ethanol (1×5 min) and dH2O (1×10 min). Antigen retrieval was done at 95-97 C. The intrinsic peroxidase activity was inhibited by incubation with 3% H₂O₂ for 10 min and nonspecific binding blocked by incubation for 1 hr with 3% goat serum at room temperature. Primary antibody against humanin (Rabbit anti-humanin, Chemicon Calif., 1:100, in 2.5% BSA-PBS) was added on specimens, and incubated over night at 4 C. After over night incubation, samples were washed with PBS for 5 min. Next, biotinylated secondary antibody was applied (goat anti-rabbit, 1:1000; Jackson ImmunoResearch Laboratories, Inc., West Grove, Pa.) for 1 hr (in the mean time we prepared ABC (Avidin-Biotin complex) solution (Vectastain kit): 1.5 ml PBS+15 μl sol A+15 μl sol B. Next, the specimens were washed with 1×PBS (to remove antibody) for 20 minutes, the last time added a little PBS-T (PBS with 0.01% Tween 20). Next, ABC solution (Santa Cruz) was applied on slides and incubated for 1 hr at room temp. After incubation, the slides were washed with 1×PBS (to remove antibody), 4 times for 20 minutes. DAB development kit (purchased from Santa Cruz) was used as instructed by the manufacturer. Next, all the slides covered with DAB were developed and counterstained with Alcian Blue (10 min), rinsed 3 times in distilled water. Next, dehydration was performed (EtOH 70%, EtOH 95%, EtOH 99%, EtOH 99%, xylene) 5 min in each. Primary antibody against humanin was neutralised with humanin peptide and used in negative control. Digital images of stained sections were collected using a Nikon Eclipse E800 microscope (Nikon Corp., Tokyo, Japan) equipped with an Olympus DP70 digital camera (Olympus Sverige AB, Solna, Sweden).

Culture of Fetal Rat Metatarsal Bones

The three middle metatarsal bone rudiments were dissected out from the paws of 20-day-old embryos (E20) (De Luca, Barnes et al. 2001). After dissection, bones were transferred to 24-well plates, one bone per well and cultured in 0.5 ml phenol-red MEM medium supplemented with 0.2% endotoxin-free fraction BSA, 1 nM β-glycerophosphate, 0.05 mg/ml ascorbic acid and 100 μg/ml penicillin/streptomycin at 37° C. under 5% CO₂ in humidified atmosphere. Medium was changed every 2-3 days. Images were captured at days 0, 2, 5, 7, 9 and 12 of culture, using a Hamamatsu C4742-95 digital camera mounted on a Nikon SMZ-U microscope. The Image-Pro plus™ (Media Cybernetics, USA) software was used to measure longitudinal growth. Metatarsal bone growth is expressed as a percentage of the length at the day of dissection (regarded as day 0). Treatment with CT (bortezomib), dexamethasone and/or HNG (S14G) was started 2-4 hours after dissection. All isolated bones were pooled and then randomly divided into the different treatment groups (4 to 6 bones per group). Each experiment was repeated at least 3 times.

Cell Culture

Mice chondrocyte cell line ATDC5 (1×10⁶), was cultured in DMEM/Ham's F12 (Invitrogen) supplemented with 5% FCS, 10 μg/ml human transferrin and 3×10⁻⁸ M (30 nM) sodium selenite until confluent (day 6), as previously reported (Owen, Miner et al. 2007). Thereafter, differentiation was induced by the addition of insulin (10 μg/ml; Sigma) and ascorbic acid (20 μg/ml) to the maintenance medium (differentiation medium). Incubation was at 37° C. in a humidified atmosphere of 95% air/5% CO2. The culture medium was changed every second day. On day 6, lipopolysacharide (LPS) 10 μg/ml for Dexa (1 μM) and HNG (10 nM, S14G) were added in DMEM/Ham's F12 only and cells were incubated further 24 hrs. Next day, the cell culture medium was collected and analyzed for Intraleukin-6 (IL-6) cytokine levels by using commercially available ELISA kit (R&D Systems, Minneapolis, Minn.) according to instructions provided from manufacturer.

Treatment of Mice with HNG (S14G) and Dexa

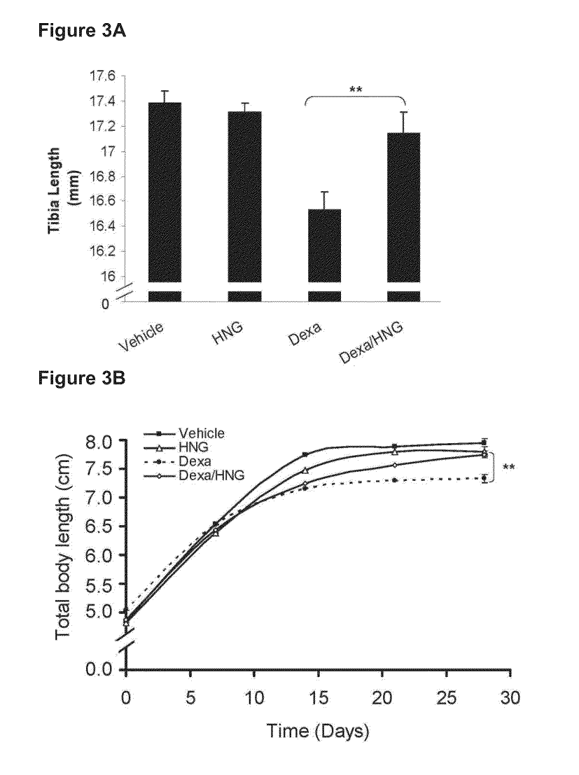

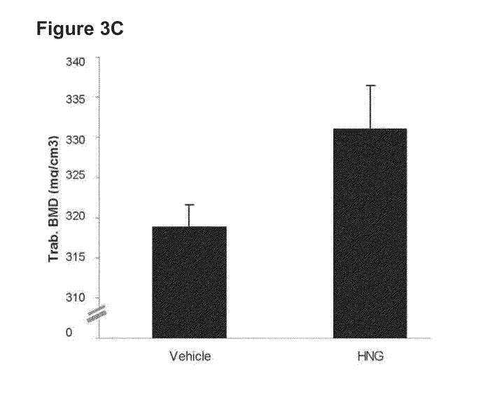

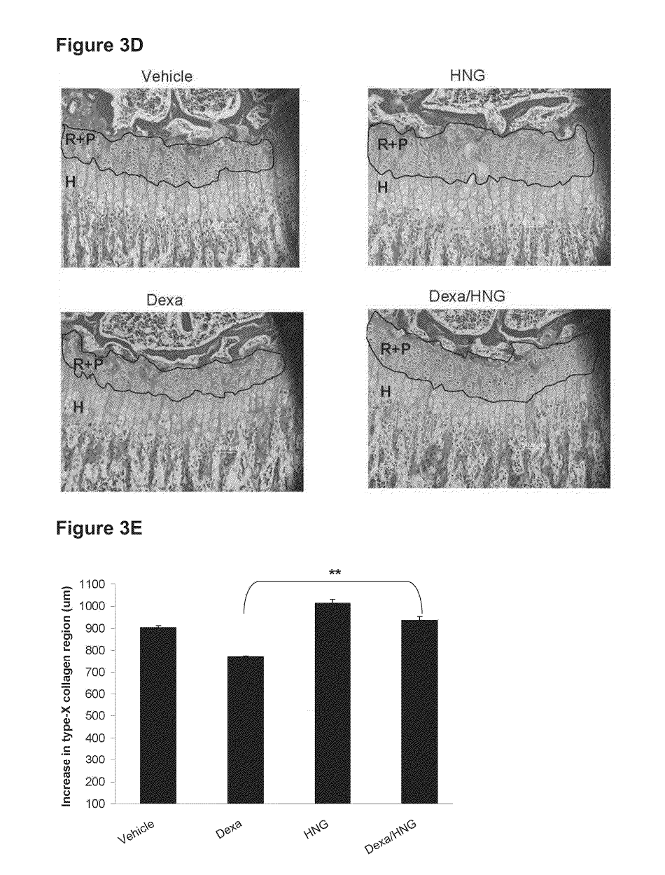

Four weeks old female mice FVB strain were obtained from Charles-River, Germany. The mice were kept in animal facility under a 12-h light, 12-h dark schedule with water ad libitum. After an adaptation period, mice were injected with Dexa dissolved in saline (20 μg/mice) subcutaneously in the neck every day for 1 week (short-term). Whereas, HNG (S14G) dissolved in saline (1 μg/mice) was injected intraperitoneally, 45 minutes prior to Dexa injection. The vehicle group received saline only. After one week of treatment/injections, animals were sacrificed and the back legs (femur and tibia) were collected. The skin and muscle from the tibia was removed and final length of tibia was measured by using electronic digital caliper as reported previously (Zaman, Menendez-Benito et al. 2007). The data shown in FIG. 3A and Example 4 indicates that HNG (S14G) protected mice from Dexa-induced growth retardation. n=9-10/group, **p<0.01.

In another study, four weeks old female mice FVB strain (Charles-river, Germany) were injected with Dexa dissolved in saline (20 μg/mice) subcutaneously in the neck every day for four week (long-term). Whereas, HNG (S14G) dissolved in saline (1 μg/mice) was injected intraperitoneally, 45 minutes prior to Dexa injection. The vehicle group received saline only. The total body length (nose to anus) was measured (centimeters, cm) manually (blinded, the cadges were coded on the day of measurment), every week. After 4 weeks of treatment, animals were sacrificed by inhalation of CO₂, tissue samples (both left and right leg) were collected for subsequent analysis. The data shown in FIG. 3B and Example 4, indicates that treatment of Dexa with HNG (314G) protected mice from Dexa-induced growth retardation n=9-10/group, **p<0.01.

Dual Energy X-ray Absorptiometry (DEXA) for BMD analysis

Areal bone mineral density (BMD; BMC/cm²) and BMC were measured by dual energy X-ray absorptiometry (DXA) using the Lunar PIXImus mouse densitometer (Wipro GE Healthcare, Madison, Wisc.). Ex vivo measurements were taken from FVB mice treated with Dexa and HNG for four weeks (for details see methods and materials). The measurements of left femur and tibia were performed on excised bones placed on a 1-cm-thick Plexiglas™ table. All bones compared were measured in the same scan (high-resolution scan with line spacing set at 0.01 cm). n=9-10/animals/group.

Growth Plate Histology

Four weeks old mice (female, FVB) were injected with Dexa dissolved in saline (20 pg/mice) subcutaneously in the neck every day for four week. Whereas, HNG (S14G) dissolved in saline (1 μg/mice) was injected intra-peritoneally, 45 minutes prior to the Dexa injection for four weeks, for details see materials and methods. The femur and tibia bones were dissected out and fixed in 4% formalin for 24 hrs, followed by decalcification in 10% EDTA and embedded in paraffin blocks. Five-micrometer-thick sections were prepared from these paraffin blocks. The sections were prepared from the tibial growth plate and stained with Van Gieson. Digital images of stained sections were collected using a Nikon Eclipse E800 microscope (Nikon Corp., Tokyo, Japan) equipped with an Olympus DP70 digital camera (Olympus Sverige AB, Solna, Sweden).

Immunohistochemistry for Type-X Collagen

Four weeks old mice (female, FVB) were injected with Dexa dissolved in saline (20 μg/mice) subcutaneously in the neck every day for four week. Whereas, HNG (S14G) dissolved in saline (1 μg/mice) was injected intra-peritoneally, 45 minutes prior to the Dexa injection. The vehicle group received saline only. The femur and tibia bones were dissected out and fixed in 4% formalin for 24 hrs, followed by decalcification in 10% EDTA and embedded in paraffin blocks. Five-micrometer-thick sections were prepared from these paraffin blocks. After one wash in dH₂O, endogenous peroxidase activity was reduced by incubation in 3% hydrogen peroxide in methanol for 5 min at room temperature. The sections were additionally treated with 5 mg/ml hyaluronidase (Sigma-Aldrich, Inc, Steinheim, Germany) for 30 min at 37° C., then blocked in 3% horse serum and incubated overnight at +4° C. with anti-collagen type-X primary antibody (Quartett GmbH, Berlin, Germany; 1:30). Secondary anti-mouse biotinylated antibody (1:300) was purchased from DakoCytomation A/S (Glostrup, Denmark). Sections were washed in TBS, incubated with avidin-conjugated peroxidase (Vector Laboratories) for 60 min, and the resulting peroxidase activity was detected using a DAB Kit (Vector Laboratories). Sections were then counterstained with Alcian blue. Measurements of the height of type-X collagen were performed within the central two-thirds of the growth plate using the Image-Pro plus™ (Media Cybernetics, USA). Minimum 4 measurements were taken from each sample and measured blindly. n=4-5/group, **p<0.01.

Treatment of Normal Mice in Vivo

Normal 5-week old male C57B and NMRI (B&K Universal, Sollentuna, Sweden) mice were housed one per cage in our animal care facility under a 12-h light, 12-h dark schedule with water ad libitum. After an adaptation period, each strain of mice were divided into two groups according to their body weight and were weight-matched into pairs to either receive 1 cycle intra peritoneal (i.p) injections of CT (bortezomib; Millennium Pharmaceuticals, USA, 1 mg/kg of body weight; 2 injections/week, 2 weeks treatment) or saline (vehicle). Body weight, food and general physical status was recorded daily during the first 20 days, and each vehicle-treated mouse was pair-fed to corresponding weight-matched CT (bortezomib)-treated mice. Mice were killed by inhalation of CO₂ 48 hrs, 45 days or 6 months after last injection or if they showed signs of poor health during the experiment. X-ray analyses where performed approximately every second week from start of treatment (represents time-point zero) to time of killing (day 56).

In Vivo Xenograft Models

Male NMRI nu/nu mice (B&K Scanbur, Sweden or Taconic, Denmark) were xenografted at the age of 4 weeks (BW 18-25 g). The mice were anesthetized with 2% fluothane (Zeneca Ltd, Macclesfield, UK) supplemented with 50% N₂O in oxygen. Tumor cells (NB (SH-SY5Y; 20×10⁶ cells/side) or MB (D-283; 10×10⁶ cells/side) in 0.1 ml medium) were implanted s.c. in the hindlegs of the animal using a 23 G needle. Tumors were measured every other day with a digital caliper, starting when they were palpable. At a tumor volume of 0.1 ml or 0.15 ml the animals were randomized into groups (1. Vehicle without tumors; 2. Vehicle with tumors; 3. HNG (S14G; 1 μg/mice); 4. CT (bortezomib; 0.8 mg/kg or 1 mg/kg); 5. HNG (S14G) 1 μg/mice+CT (bortezomib; 0.8 mg/kg or 1 mg/kg), stratified by their bodyweight and treatment started. Treatment regime was the same as in clinical settings with 2 injections/week; 2 weeks treatment (see FIG. 1, Group 1). HNG (S14G) was injected 40 min before the CT (bortezomib) injection. Tumor volume was calculated by the formula 0.44×length×width×width (Wassberg, Pahlman et al. 1997). The animals were housed in an isolated room at 24° C. with a 12-h day/night cycle with water ad libitum and restricted food pellets from start of treatment. The animal weight and general appearance were recorded every day throughout the experiments. CT (bortezomib) was administered intraperitoneally (i.p; 1.0 mg/kg) or intravenously (i.v; 0.8 mg/kg) and/or HNG (S14G) or saline (vehicle) was administered intra peretoneal (i.p). The experiments were terminated before the tumors reached a volume of 2 mL or if the animal showed signs of poor health. Some animals had to be killed before completion of the experimental endpoint. Bone growth was followed by X-ray analyses at start of treatment (day 0) and at time of killing (day 13, 48 hrs after last injection). Blood and tissue samples were collected for subsequent analysis; tibia, femur and tumors were fixed in 4% formaldehyde in 0.1M phosphate buffer, pH 7.3, for 24 hrs at 4° C. After fixation, femur and tibia were measured with digital calipers and decalcified in 5% EDTA and then paraffin embedded.

### EXAMPLE 1

### Humanin is Expressed in Human Chondrocytes/Growth Plate Cartilage

In the FIG. 1B, biopsies of human growth plate (obtained from a 14 year old boy who was operated for removal of growth plate by an orthopedic surgeon) were used to investigate the expression of humanin in chondrocytes. Humanin was found widely expressed in chondrocytes, mainly in the resting and proliferative zones. In negative control, primary antibody against humanin was neutralised with humanin peptide, showed no staining of humanin.The data shows that humanin is expressed in human chondrocytes providing a first ever evidence for its expression in human chondrocytes. The wide expression of humanin in chondrocytes suggests that humanin may be invloved in different cellular activities in chondrogenesis. Moreover, the strong presence of humanin in the resting, proliferative and hypertrophic zones further suggests that it may also be involved in the modulation of bone growth.

### EXAMPLE 2

### Growth Promoting/Protective Effects of Humanin Analog HNG (S14G) and Colivelin(Col) on Longitudinal Bone Growth in Cultured Fetal Rat Metatarsal Bones

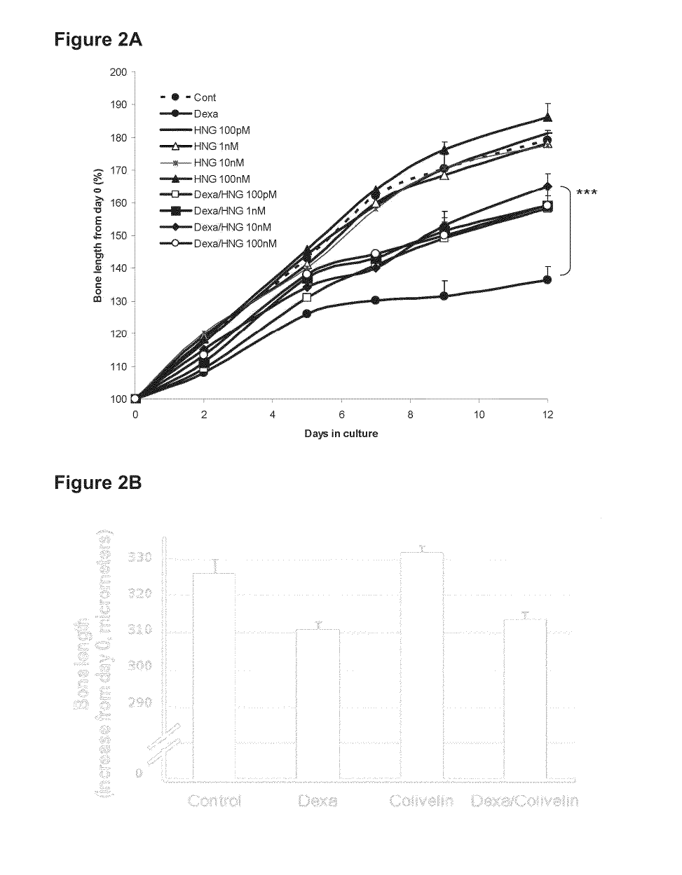

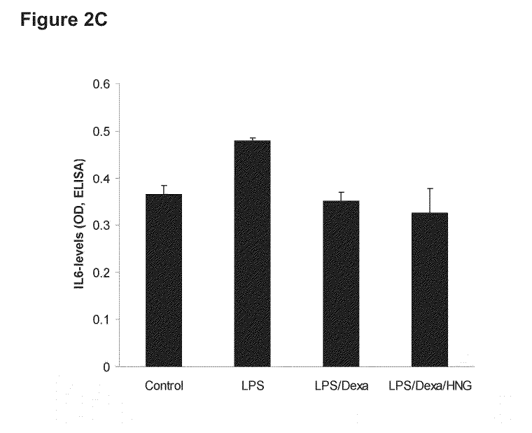

The data shown in FIG. 2A indicates novel growth promoting/protective effects of humanin analog HNG (S14G) on longitudinal bone growth in cultured fetal rat metatarsal bones. Dexa (1 μM) treatment induced growth retardation but combined treatment with Dexa and different doses of HNG (S14G) significantly rescued the bones from Dexa-induced growth retardation. Briefly, fetal rat metatarsal bones were pre-treated with different concentrations (mentioned in the figure) of HNG (S14G), 30 min prior to addition of Dexa. Interestingly, HNG (S14G), in combination with Dexa, not only blocked negative side effects of Dexa on bone growth but also improved bone growth by itself (at 100 nM on day 12) above control. These results suggest a direct effect of HNG (S14G) on bone growth and its growth promoting effects by itself further suggest its crucial role in chondrogensis. FIG. 2B shows that colivelin, a humanin derivative, exerts some stimulatory effect on longitudinal growth of fetal rat metatarsal bones cultured for 12 days. When, colivelin (100 μM) was combined with Dexa (1 μM), it had some protective effect against Dexa-induced bone growth retardation. These data suggest that colivelin has the capacity to positively influence bone growth. (n=4-6 bones/group)

### EXAMPLE 3

### Humanin Analog HNG (S14G) Does Not Alter the Anti-Inflammatory Effects of Dexa, When Given in Combination

FIG. 2C. Since Dexa is widely used in inflammatory conditions to suppress/block inflammation, it was important to investigate whether combined treatment of HNG (S14G) with Dexa does not alter/interfere with the anti-inflammatory effects of Dexa. Therefore, ATDC5 chondrocytes (mice) were cultured and treated with lipopolysacharide (LPS, 10 μg/ml) for 24 hrs to induce inflammation. As shown in FIG. 2C, the levels of the cytokine IL-6 were increased in the culture medium of LPS treated cells, whereas Dexa (1 pM) treatment completely suppressed/blocked IL-6 production (n=2-3 observations/group). Importantly, the combined treatment of Dexa and HNG (S14G) did not alter the anti-inflammatory effects of Dexa, and the levels of IL-6 cytokine remained same as in the LPS+Dexa group. These data suggest that the combination of Dexa and HNG (S14G) is a proper treatment strategy to block the negative side-effects of Dexa without affecting the desired anti-inflammatory effects of Dexa.

### EXAMPLE 4

### Humanin Analog HNG (S14G) Rescues Mice From Dexamethasone-Induced Growth Retardation

Since it was shown in previous figures that HNG rescues metatarsal bones from Dexa-induced growth retardation, this was verified in a mouse model of Dexa-induced growth retardation. As shown in FIG. 3A, FVB mice received Dexa (20 μg/mice/day) and HNG (1 μg/mice/day) injections for 1 week (group 1) and 4 weeks (group 2); for details see material and methods. HNG injections were given 30 min prior to Dexa injections. In group 1 (FIG. 3A), Dexa significantly induced growth retardation, whereas HNG (S14G) when given in combination with Dexa rescued mice from Dexa-induced growth retardation. After this, we next asked whether HNG (S14G) has any protective effects on bone growth after long-term treatment with Dexa (FIG. 3A). To investigate this, we treated mice for 4 weeks (group 2) with Dexa (20 μg/mice/day) and HNG (1 μg/mice/day). Mice treated with Dexa alone were significantly growth retarded, compared to vehicle as expected (FIG. 3B). But interestingly, HNG (S14G) completely rescued mice from Dexa-induced growth retardation even after 4 weeks of Dexa-treatment. These data suggest that HNG (S14G) is capable of blocking negative side-effects of Dexa on longitudinal bone growth by exerting positive effects on chondrogensis which is altered due to Dexa treatment. In FIG. 3B, total growth of the animals, nose to anus length (cm, manually) was analyzed in vehicle, Dexa, HNG (S14G) and Dexa/HNG (S14G) treated animals after four weeks of injections. **p<0.01 by 1-way ANOVA analyses, n=9-10/animals/group).

### EXAMPLE 5

### Humanin Analog HNG (S14G) Increases the Bone Mineral Density (BMD) in Mice

In the light of the above findings where it was observed rescuing effects of HNG (S14G) on bone growth both in vitro and in vivo, it was analyzed whether HNG (S14G) has the capacity to regulate BMD. As shown in FIG. 3C, it was identified a trend towards increased BMD in HNG (S14G; 1 μg/mice/day, for four weeks) treated mice, compared to vehicle. Trabecular BMD in tibia (mg/cm³) of four-week old FVB mice treated for 4 weeks was measured by using DEXA (see protocol in material and methods). These data suggest that HNG (S14G) has the capacity to positively regulate BMD thereby reducing the risk of bone fractures.

### EXAMPLE 6

### Humanin Analog HNG (S14G) Stimulates Chondrogensis by Regulating Resting and Proliferating Chondrocyte Zone (R+P)

Since a positive effect of HNG (S14G) on bone growth was identified, different zones of chondrocytes were analyzed (resting, proliferative and hypertrophic) in growth plate cartilage, to identify visible changes in these zones. Interestingly, as shown in the drawings in FIG. 3D it was found that that the zone containing resting and proliferating chondrocytes (R+P) is a primary key target of HNG (S14G) within growth plate cartilage, because this zone appears to be larger in HNG (S14G) treated animals than in all other groups. In Dexa treated animals, the R+P zone appears smaller but the rescuing effect of HNG (S14G) on the R+P zone can be clearly seen in animals which had received HNG (S14G)/Dexa, as the zone appears to be larger than in the Dexa only treated group. These findings suggest that HNG (S14G) has the capacity to maintain the integrity of the growth plate chondrocyte zones which play a crucial role in chondrogenesis and bone growth. These micropgraphas also explain the longitudinal bone growth promoting effects by HNG (S14G) alone or in combination with Dexa.

### EXAMPLE 7

### Humanin Analog HNG (S14G) Positively Regulates Chondrocyte Differentiation Process by Regulating Type-X Collagen in the Mouse Growth Plate

Type X collagen is synthesized/produced specifically by hypertrophic chondrocytes, and this specificity is primarily regulated at the level of transcription. Recently, a mutation in type-X collagen has been reported in Schmid metaphyseal chondroplasia, suggesting an important role of type-X collagen for normal bone growth. Moreover, it has been reported that Dexa alters the chondrocyte differentiation process and also impairs type-X collagen expression in chondrocytes. Therefore, the status of type-X collagen expression was examined in tibia growth plates from animals treated with HNG (S14G) and Dexa, alone or in combination. Briefly, four-week old mice (female, FVB) were injected with Dexa (20 μg/mice) subcutaneously in the neck and HNG (S14G) (1 μg/mice) every day for four week. Whereas, HNG (S14G) was injected intra-peritoneally, 30 minutes prior to the Dexa injection. The findings represented in FIG. 3E clearly show that HNG (S14G) counteracts the negative effects of Dexa on type-X collagen expression (see material and methods). Interestingly, the type-X collagen was significantly increased in Dexa/HNG (S14G) treated animals, compared to Dexa alone (p<0.01 in Dexa/HNG (S14G) vs Dexa alone). Furthermore, type-X collagen was also increased in HNG (S14G) only treated animals, which further supports the positive role of humanin analogs on chondrocytes differentiation. These data suggests that HNG (S14G) plays a key role in maintenance of growth plate chondrocytes, because production of type-X collagen within growth plate provides a favorable environment for proliferation and differentiation to the growth plate chondroctes.

### EXAMPLE 8

### Growth Retardation Caused by CT (bortezomib) in Vitro

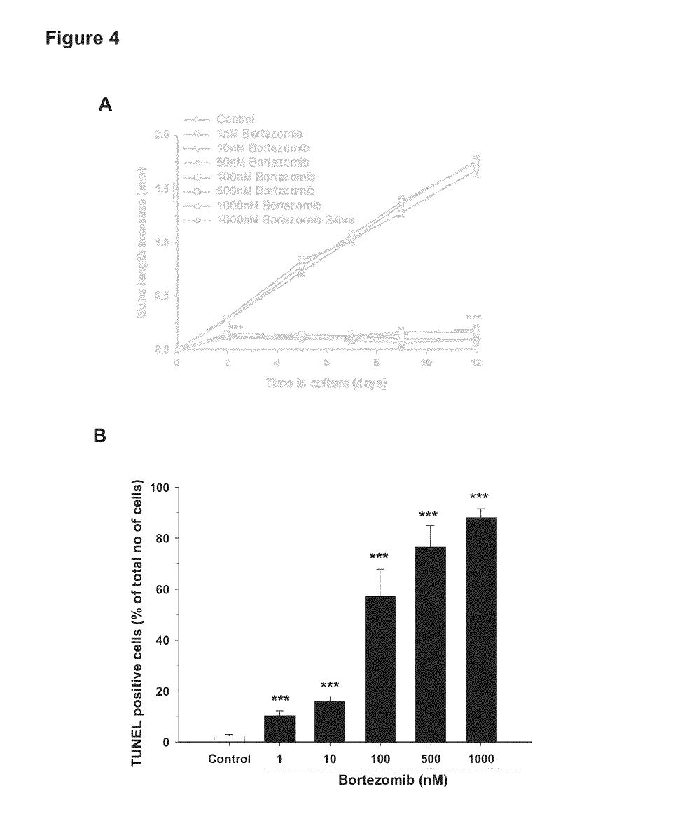

CT (bortezomib) is a cytotoxic drug known to cause critical alterations in cells/tissues and disturb the cellular homeostasis. To test the effect of CT (bortezomib) on chondocytes and longitudinal bone growth, the established in vitro model of fetal rat metatarsal bones was used. These bones were collected as described in material and methods, cultured and treated with different concentrations of CT (bortezomib) under sterile conditions for up to 12 days. Significant growth suppression was observed as early as 2 days after exposure to CT (bortezomib) at 50 nM or higher concentrations and then sustained throughout the experiment (FIG. 4A; ***p<0.001 vs. control). Interestingly, when metatarsals were exposed to 1000 nM CT (bortezomib) for only 24 hrs and then cultured in control medium for up to 12 days, this was sufficient to cause permanent growth arrest (FIG. 4A, dotted line; ***p<0.001 vs. control). Apoptosis was analyzed in whole growth plate sections of 12-day cultured rat metatarsal bones (perichondrium excluded) by applying the TUNEL technique to detect DNA fragmentation. For all concentrations of CT (bortezomib) tested (1-1000 nM), a significant dose-dependent increase in chondrocyte apoptosis was observed (FIG. 4B; ***p<0.001 vs. control bones). In light of these results it is clear that (CT) bortezomib severely impairs longitudinal bone growth and affects cell death (increased TUNEL-positive cells), in this in vitro model.

### EXAMPLE 9

### CT (Bortezomib) Impairs Longitudinal Bone Growth in Vivo

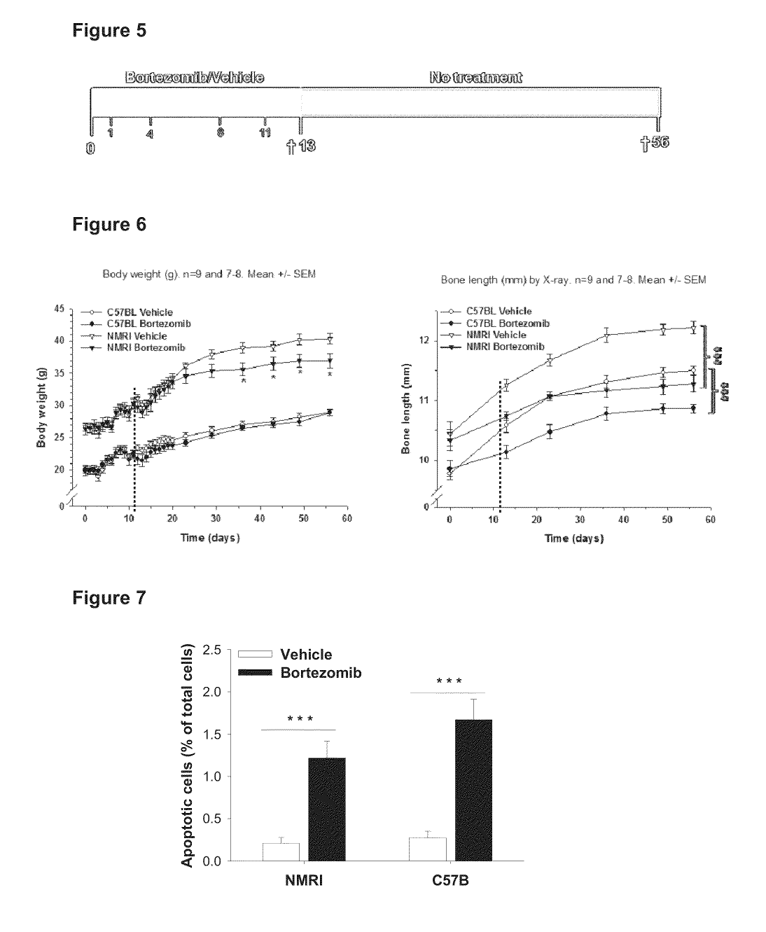

FIG. 5 illustrates a schematic representation of the experimental design for the in vivo studies in C57B and NMRI mice. Both strains of mice were housed one per cage with water ad libitum. At start of treatment, mice were randomized into two groups based on their body weight and were weight-matched into pairs to either receive 1 cycle of intraperitoneal (i.p.) injected CT (bortezomib, 1 mg/kg) or vehicle at the indicated time-points; day 1, 4, 8 and 11. Each vehicle treated mouse was pair-fed to a corresponding weight-matched CT (bortezomib) treated mouse. Mice were killed (t) on day 13 (48 hrs post treatment) or on day 56 (45 days post treatment). X-ray analyses where performed approximately every second week from start of treatment (represents time-point zero) to time of killing (day 13 or 56). Bone lengths (mm) of femur were analyzed in vehicle- and CT (bortezomib) treated animals. Each group included 7-10 mice.

Body weight was measured in both strains of mice every day until day 20 after start of treatment, and once per week thereafter. From day 36 until day 56 after start of treatment there is a significant difference in body weight between NMRI CT (bortezomib; filled triangles) and vehicle-treated mice (open triangles); *p<0.05; FIG. 6A. However, there is no difference in body weight between CT (bortezomib; filled circles) and vehicle- (open circles) treated C57BL mice. The vertical hatched line in FIGS. 6A and B indicates the last injection on day 11. In light of the growth inhibiting effect on metatarsal bones in vitro and the association of many other cytotoxic drugs, the effect on bone growth was investigated in vivo. Normal 5-week old male C57B and NMRI mice were weigh-matched into pairs to either receive 1 cycle intra peritoneal (i.p) injections of CT (bortezomib) at a clinical relevant dose (1mg/kg of body weight; 2 injections/week, 2 weeks treatment) or saline (vehicle). One group were killed 48 hrs after last injection (day 13) to study the direct effects on bone growth and the growth plate chondrocytes. The second group of animals were followed another 45 days without any treatment and then killed (day 56, 45 days post treatment) to look at the long-term effect and if there was any catch-up growth. Bone growth was followed longitudinally by X-ray analyses throughout the experiment. Remarkably, considering the relatively short duration of treatment, bone length were found to be significantly shorter in comparison to vehicle-treated mice (day 13; ***p<0.001 for both strains of mice vs vehicle; FIG. 6B). More interestingly, these effects were permanent, so even after the 45 day follow-up period, CT (bortezomib)-treated mice were still significantly shorter (***p<0.001; FIG. 6B). These results suggests that only 1 cycle of the CT (bortezomib) is enough to cause impairment of chondrogenesis which results in severe long-term side effects on linear bone growth. Same results were obtained in two different strains of mice, further strengthening the observed effect and also showing that is is not strain-specific.

### EXAMPLE 10

### CT (Bortezomib) Treatment Induced Chondrocyte Apoptosis in Vivo

Cell death is a well known and desirable phenomenon in the anti-cancer therapy. However, side-effects of the treatment in other tissues and cells is not uncommon. To further investigate the underlying mechanism(s) to the observed growth impairment in vivo we studied apoptosis (by the TUNEL method). Apoptosis was quantified in paraffin embedded growth plate sections of CT (bortezomib) and vehicle treated C57B and NMRI mice sacrificed on day 13 of the study (48 hrs post treatment). In each strain of mice, CT (bortezomib) caused a significant induction of chondrocyte apoptosis compared to vehicle; ***p<0.001 respectively; FIG. 7. These results suggests that one of the underlying mechanisms of CT (bortezomib)-induced growth retardation in vivo is increased chondrocyte apoptosis.

### EXAMPLE 11

### CT (Bortezomib) Induced Apoptosis in Cultured Human Growth Plate Cartilage

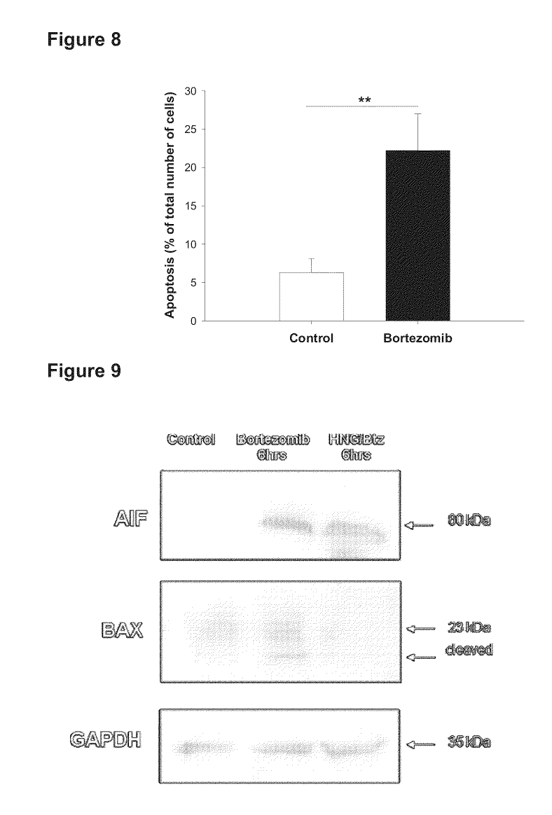

Biopsies from the proximal tibia and distal femur growth plates were collected in 5 pubertal children (3 boys and 2 girls) undergoing epiphyseal surgery for different medical conditions (4 patients with constitutional tall stature and 1 patient with leg length discrepancy). Biopsies were cut into thin slices under microscope and cultured for 24 hrs in the presence of 1000 nM CT (bortezomib). After fixation and sectioning, growth plates were analyzed using the TUNEL assay to determine the levels of chondrocyte apoptosis. FIG. 8 shows the quantification of apoptosis in paraffin embedded human growth plate cartilage biopsies. CT (bortezomib) caused a significant increase in apoptosis (22.2±4.4% TUNEL positive cells vs. 6.3±1.8% in control; **p≦0.01, n=5 patients), which was mainly observed in stem-like and early proliferative chondrocytes. Our confirmation that human growth plate cartilage is also highly sensitive to bortezomib-induced cytotoxicity is of considerable clinical importance. Based on these data, we stress the importance of identifying cytoprotective strategies to minimize any undesired side-effects on linear bone growth when proteasome inhibitors are given to individuals with childhood cancers.

### EXAMPLE 12

### Humanin Analog HNG (S14G) Protects Chondrocytes From CT (Bortezomib)-Induced Up-Regulation of Pro-Apoptotic Proteins in Vitro

Western immunoblotting to study protein expression was performed in the rat C5.18 chondrocytic cell line after treatment with 1000 nM CT (bortezomib) or 100 nM humanin analog HNG (S14G)/1000 nM CT (bortezomib (Btz)) for 6hrs. Results in FIG. 9 show that CT (bortezomib) induce up-regulation and activation of the two pro-apoptotic proteins, apoptosis inducing factor (AIF, 60 kDa) and BAX (23 kDa and its cleaved fragment). By adding humanin analog HNG (S14G), the up-regulation and activation of the two pro-apoptotic proteins were suppressed. These data suggests that humanin analog HNG (S14G) interferes with these pro-apoptotic proteins and thereby blocks the apoptosis-inducing pathway(s). As an internal control the house-keeping gene, GAPDH (35 kDa) was used. The experiment was repeated twice.

### EXAMPLE 13

### Humanin Analog HNG (S14G) Protects Fetal Rat Metatarsal Bones From Ct (Bortezomib)-Induced Growth Retardation in Vitro

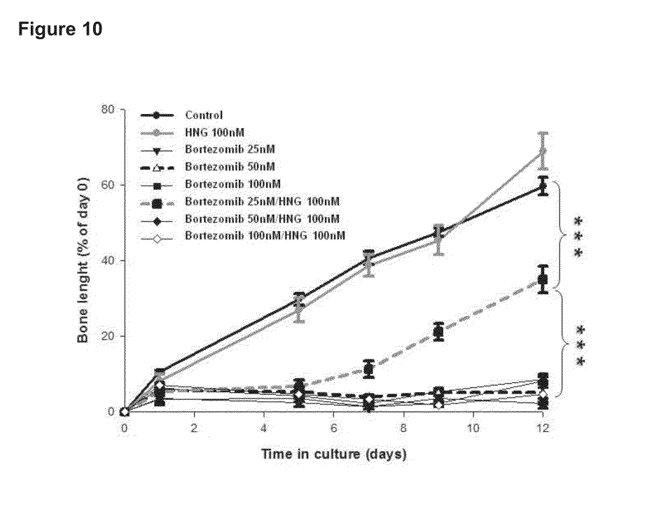

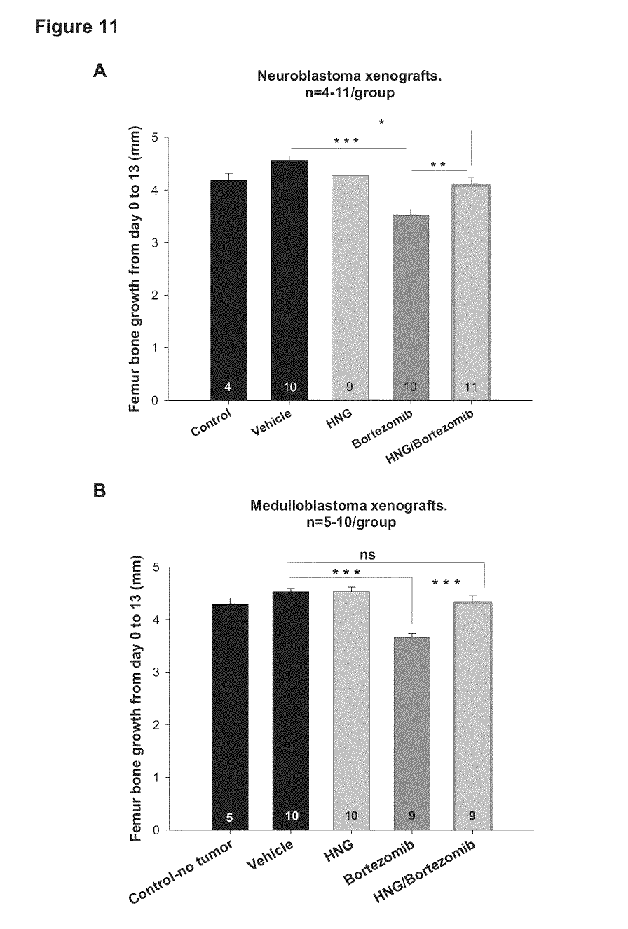

In an attempt to block and prevent the negative effects on growth after CT (bortezomib)-treatment, recently discovered humanin analog HNG (S14G) was tested in vitro in fetal rat metatarsal bones. As shown in FIG. 10, CT (bortezomib), at all concentrations tested (25-100 nM) completley abolished growth. Surprisingly, in this in vitro model, it was possible to rescue the growth of bones up to 50% by adding the humanin analog HNG (S14G) to the culture medium in combination with CT (25 nM bortezomib, ***p<0.001). However, at higher doses of CT (bortezomib; 50 nM and above) used, humanin analog HNG (S14G) was not able to rescue the growth impairment. Another observation of humanin analog HNG (S14G) is that it not only blocked the negative side effects of CT (bortezomib) but also showed a trend to promote bone growth on its own at 100 nM (on day 12). Taken together, these results suggest that the humanin analog HNG (S14G) is a strong candidate to be used in combination with CT (bortezomib) to rescue and prevent unwanted side effects on linear bone growth and might also improve bone growth by itself.

### EXAMPLE 14

### Humanin Analog HNG (S14G) Rescues Mice From CT (Bortezomib)-Induced Growth Retardation

NMRI nude mice harboring either human- neuroblastoma (SK-N-BE(2)) or medulloblastoma (D-283) cancer xenografts were randomized to either receive intraperitoneal (i.p) injections of humanin analog HNG (S14G; 1 μg/mice) and/or CT (bortezomib; lmg/kg) or saline (vehicle) over 11 days (injections on day 1, 4, 8 and 11, same as in the clinic) and killed on day 13 (48 hrs after last injection). All animals were placed one and one per cage and food-restricted (given an adjusted amount of food each day) to be able to conclude that any observed effect(s) was due to the drug(s) and not because of differences in nutrition/food intake. Bone growth was followed by X-ray analyses taken before start of treatment (time-point 0) and at time of killing (day 13, 48 hrs after last injection). Remarkably, the humanin analog HNG (S14G) was able to rescue and prevent the undesired side effects of CT (bortezomib) on linear bone growth in two different models of human xenografts (neuroblastoma and medulloblastoma, FIGS. 11A and B respectively, ns=non significant; *p<0.05; ***p<0.001). These findings provide direct in vivo evidence that the humanin analog HNG (S14G) can rescue CT (bortezomib) induced growth retardation.

### EXAMPLE 15

### Humanin Analog HNG (S14G) Does Not Alter the Anti-Cancer Effects of CT (Bortezomib), When Given in Combination

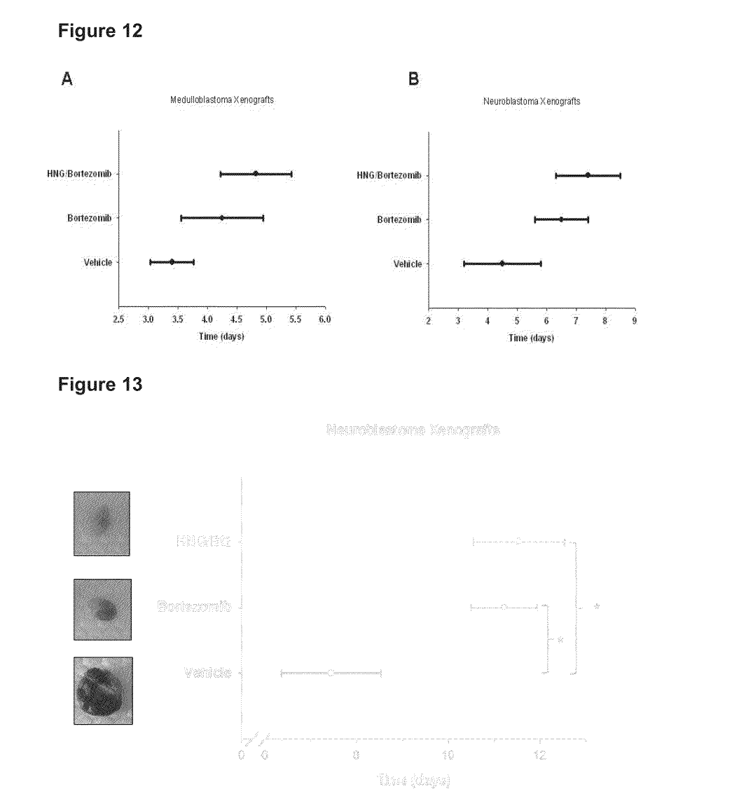

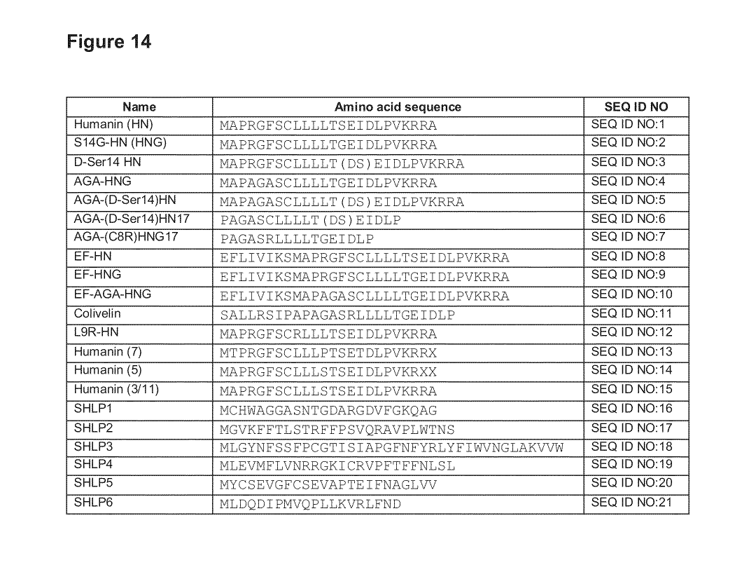

Since bortezomib is a CT drug, used for treatment of cancers, it is essential to rule out any possible interference between humanin analog HNG (S14G) and CT (bortezomib). To test this hypothesis, two different human tumor xenograft models were created by injection of either medulloblastoma (D-283) cells or neuroblastoma (SK-N-BE(2)) cells (to NMRI nude mice). The mice were thereafter treated with intraperitoneal (i.p.) injections of CT (bortezomib), combination of CT (bortezomib)+humanin analog HNG (S14G) or saline only (vehicle). Tumor volumes were measured with caliper every day from start of treatment until end of treatment (killing, day 13) and also weighed after removal. Our results indicate that the humanin analog HNG (S14G) does not interfere with the anti-cancer effect of CT (bortezomib; FIGS. 12A and B). An important conclusion from this experiment, shown in two different human xenograft models (medulloblastoma and neuroblastoma), is that the humanin analog HNG (S14G) does not interfere with the anti-cancer effect of CT (bortezomib).

### EXAMPLE 16

### Humanin Analog HNG (514G) Does Not Alter the Anti-Cancer Effects of CT (Bortezomib) in Neuroblastoma When Given in Combination

Doubling time of tumors in neuroblastoma (SK-N-BE(2)) tumor xenografts treated with vehicle, CT (bortezomib) or the combination CT (bortezomib)/humanin analog HNG (S14G), n=9-11/group. A significant reduction in tumor doubling time was observed with both CT (bortezomib) and the combination compared to vehicle treated mice (*p<0.05 respectively; FIG. 13). Treatment with CT (bortezomib) was given in a clinically relevant dose (0.8 mg/kg, 120 μl), with same route and administration as in patients, intravenously (i.v.) on day 1, 4, 8 and 11. Humanin analog HNG (S14G) were given intraperitonealy (i.p., 1 μg/mouse/injection, 200 μl) and vehicle received saline both i.v. (120 μl) and i.p. (200 μl). Also these results conclude that the humanin analog HNG (S14G) does not interfere with the anti-cancer effect of CT (bortezomib), instead it might even improve it.

1. Humanin or a humanin-like peptide (HLP) or a fragment, analog or derivative thereof, for use in the treatment and/or prevention of a bone- or cartilage disorder.

2. The peptide for use according to claim 1, wherein said disorder is drug-induced.

3. The peptide for use according to claim 1 or 2, wherein said peptide comprises an amino acid sequence selected from SEQ ID NO:1 to SEQ ID NO:21, as well as peptide comprising an amino acid sequence having at least 60%, at least 65%, at least 70%, at least 75%, at least 80%, or at least 85% sequence identity to the peptide sequences defined in SEQ ID NO:1 to SEQ ID NO:21.

4. The peptide for use according to claim 1 or 2, wherein said peptide comprises an amino acid sequence selected from SEQ ID NO:1 to SEQ ID NO:21.

5. The peptide for use according to claim 1 or 2, wherein said peptide comprises an amino acid sequence of SEQ ID NO:2.

6. The peptide according to claims 1 to 5, wherein said bone- or cartilage disorder is either a primary or secondary bone- or cartilage disorder.

7. The peptide for use according to any one of claims 1 to 6, wherein said disorder is drug-induced from drugs used in the treatment of rheumatological disorders, respiratory disorders, gastrointestinal disorders, cardiovascular disorders, endocrinological disorders, cancer, neurodegenerative disorders, kidney disorders, liver disorders, dermatologic disorders, allergic disorders, inflammatory disorders, metabolic disorders, undesired immune response, undesired inflammatory response, obesity or diabetes.

8. The peptide for use according to any one of claims 1 to 7, for use in the treatment and/or prevention of drug-induced bone growth impairment, short stature and osteoporosis.

9. The peptide for use according to any one of claims 1 to 8, wherein said peptide improves bone growth.

10. The peptide for use according to any of the claims 1 to 8, wherein said peptide prevents development of osteoporosis.

11. The peptide for use according to claim 7, wherein said drug is an anti-inflammatory drug.

12. The peptide for use according to claim 11, wherein said anti-inflammatory drug is a glucocorticoid drug.

13. The peptide for use according to claim 12, wherein said glucocorticoid drug is selected from hydrocortisone, hydrocortisone buteprate, hydrocortisone butyrate, budesonide, ciclesonide, cortisone acetate, deflazacort, medrysone, tixocortol, cloprednol, halcinonide, pregnadiene, rimexolone, flunisolide, triamcinolone, amcinonide, fluocinolone acetonide, fluocinonide, fluorometholone, clocortolone, diflucortolone, fluocortin, desoximetasone, prednisone, prednisolone, methylprednisolone, methylprednisolone aceponate, prednicarbate, prednylidene, desonide, fluprednisolone, difluprednate, fluperolone, meprednisone, dexamethasone, betamethasone, triamcinolone, beclometasone, dobetasone, diflorasone, halometasone, ulobetasol, fludrocortisone acetate, beclomethasone dipropionate, beclomethasone monopropionate, paramethasone, alclometasone, fluclorolone, flumetasone, fluprednidene, triamcinolone, flunisolide, cortivazol, fluticasone, fluticasone propionate, fluticasone furoate, loteprednol, fludroxycortide, fomiocortal and mometasone furoate.

14. The peptide for use according to claim 12, wherein said glucocorticoid drug is dexamethasone.

15. The peptide for use according to claim 7, wherein said drug is an anti-cancer drug.

16. The peptide for use according to claim 15, wherein said anti-cancer drug is a proteasome inhibitor.

17. The peptide for use according to claim 16, wherein said proteasome inhibitor is selected from bortezomib, MLN9708, MLN273, MLN519, NPI-0052, disulfiram, ritonavir, lactacystin, CEP-18770, carfilzomib, PS-519, MG132, MG 262, ALLN, fellutamide B, tyropeptin A, omuralide, salinosporamide A, eponeomycine, epoxomicin, TMC-95A, syringolin A, glidobactin A, TMC-95 analogs, NLVS and ZLVS.

18. The peptide for use according to claim 16, wherein said proteasome inhibitor is bortezomib.

19. The peptide for use according to claim 7, wherein said drug is a GnRH agonist drug, such as leuprolide, buserelin, nafarelin, histrelin, goserelin or deslorelin.

20. The peptide for use according to claim 7, wherein said drug is a GnRH antagonist drug, such as cetrorelix, ganirelix, abarelix and degarelix.

21. The peptide for use according to any one of claims 1 to 20, wherein said peptide is administered in combination with another drug known to induce a bone- or cartilage disorder.

22. The peptide for use according to claim 21, wherein said another drug is selected from an anti-inflammatory drug, a selective estrogen receptor modulator drug, an anti-androgen drug, an aromatase inhibitor drug, a GnRH antagonist drug, a GnRH agonist drug, and an anti-cancer drug.

23. The peptide for use according to claim 21, wherein said another drug is an anti-inflammatory drug.

24. The peptide for use according to claim 23, wherein said anti-inflammatory drug is a glucocorticoid drug.

25. The peptide for use according to claim 24, wherein said glucocorticoid drug is selected from hydrocortisone, hydrocortisone buteprate, hydrocortisone butyrate, budesonide, ciclesonide, cortisone acetate, deflazacort, medrysone, tixocortol, cloprednol, halcinonide, pregnadiene, rimexolone, flunisolide, triamcinolone, amcinonide, fluocinolone acetonide, fluocinonide, fluorometholone, clocortolone, diflucortolone, fluocortin, desoximetasone, prednisone, prednisolone, methylprednisolone, methylprednisolone aceponate, prednicarbate, prednylidene, desonide, fluprednisolone, difluprednate, fluperolone, meprednisone, dexamethasone, betamethasone, triamcinolone, beclometasone, clobetasone, diflorasone, halometasone, ulobetasol, fludrocortisone acetate, beclomethasone dipropionate, beclomethasone monopropionate, paramethasone, alclometasone, fluclorolone, flumetasone, fluprednidene, triamcinolone, flunisolide, cortivazol, fluticasone, fluticasone propionate, fluticasone furoate, loteprednol, fludroxycortide, formocortal and mometasone furoate.

26. The peptide for use according to claim 25, wherein said glucocorticoid drug is dexamethasone.

27. The peptide for use according to claim 21, wherein said drug is an anti-cancer drug.

28. The peptide for use according to claim 27, wherein said anti-cancer drug is a proteasome inhibitor.

29. The peptide for use according to claim 28, wherein said proteasome inhibitor is selected from bortezomib, MLN9708, MLN273, MLN519, NP1-0052, disulfiram, ritonavir, lactacystin, CEP-18770, carfilzomib, PS-519, MG132, MG 262, ALLN, fellutamide B, tyropeptin A, omuralide, salinosporamide A, eponeomycine, epoxomicin, TMC-95A, syringolin A, glidobactin A, TMC-95 analogs, NLVS and ZLVS.

30. The peptide for use according to claim 28, wherein said proteasome inhibitor is bortezomib.

31. The peptide for use according to claim 21, wherein said drug is a GnRH agonist drug, such as leuprolide, buserelin, nafarelin, histrelin, goserelin or deslorelin.

32. The peptide for use according to claim 21, wherein said drug is a GnRH antagonist drug, such as cetrorelix, ganirelix, abarelix and degarelix.

33. The peptide for use according to claim 21, wherein said drug is selected from leuprolide, buserelin, nafarelin, histrelin, goserelin, deslorelin, cetrorelix, ganirelix, abarelix, degarelix, letrozole, anastrozole, exemestane, vorozole, formestane, fadrozole, aminoglutethimide, testolactone, tamoxifen, afimoxifene, 4-hydroxytamoxifen, arzoxifene, bazedoxifene, clomifene, femarelle, lasofoxifene, ormeloxifene, raloxifene, tamoxifentoremifene, spironolactone, cyproterone acetate, flutamide, nilutamide, bicalutamide, ketoconazole, finasteride, dutasteride, cyclophosphamide, mechlorethamine, uramustine, melphalan, chlorambucil, ifosfamide, carmustine, lomustine, streptozocin, busulfan, thiotepa, cisplatin, carboplatin, nedaplatin, oxaliplatin, satraplatin, triplatin tetranitrate, procarbazine, altretamine, dacarbazine, mitozolomide, temozolomide, imatinib mesylate, erlotinib, gefitinib, sunitinib, roscovitine, bevacizumab, rapamycin, cyclosporin A, tacrolimus, 5-fluorouracil, methotrexate, etoposide, doxorubicin, actinomycine, vitamin A acid, 13-cis-retinoic acid, 2-chlorodeoxyadenosine, 5-azacitidine, 5-fluorouracil, 6-mercaptopurine, 6-thioguanine, abraxane, accutane, actinomycin-d, adriamycin, adrucil, afinitor, agrylin, ala-cort, aldesleukin, alemtuzumab, alitretinoin, alkaban-aq, alkeran, all-transretinoic acid, alpha interferon, altretamine, amethopterin, amifostine, aminoglutethimide, anagrelide, anandron, anastrozole, arabinosylcytosine, ara-c, aranesp, aredia, arimidex, aromasin, arranon, arsenic trioxide, asparaginase, avastin, azacitidine, bendamustine, bevacizumab, bexarotene, bicalutamide, blenoxane, bleomycin, busulfan, busulfex, calcium leucovorin, campath, camptosar, camptothecin-11, capecitabine, carac, carboplatin, carmustine, carmustine wafer, casodex, cerubidine, cetuximab, chlorambucil, cisplatin, citrovorum factor, cladribine, cortisone, cosmegen, cyclophosphamide, cytadren, cytarabine, cytarabine liposomal, cytosar-U, cytoxan, dacarbazine, dacogen, dactinomycin, darbepoetin alfa, dasatinib, daunomycin, daunorubicin, daunorubicin hydrochloride, daunorubicin liposomal, daunoxome, decadron, decitabine, delta-cortef, deltasone, denileukin diftitox, depocyt, dexamethasone acetate, dexamethasone sodium phosphate, dexasone, dexrazoxane, dhad, dic, diodex, docetaxel, doxil, doxorubicin, doxorubicin liposomal, droxia, duralone, efudex, eligard, ellence, eloxatin, elspar, emcyt, epirubicin, epoetin alfa, erbitux, erlotinib, erwinia L-asparaginase, estramustine, ethyol, etopophos, etoposide, etoposide phosphate, eulexin, everolimus, evista, exemestane, fareston, faslodex, femara, filgrastim, floxuridine, fludara, fludarabine, fluoroplex, fluorouracil, fluorouracil (cream), fluoxymesterone, flutamide, folinic acid, fulvestrant, gefitinib, gemcitabine, gemtuzumab ozogamicin, gemzar, gleevec, gliadel wafer, goserelin, halotestin, herceptin, hexadrol, hexalen, hexamethylmelamine, hycamtin, hydrea, hydrocort acetate, hydrocortisone, hydrocortisone sodium phosphate, hydrocortisone sodium succinate, hydrocortone phosphate, hydroxyurea, ibritumomab, ibritumomab tiuxetan, idamycin, idarubicin, ifex, ifosfamide, imatinib mesylate, imidazole carboxamide, interferon alfa, interleukin-2, interleukin-11, intron A (interferon alfa-2b), iressa, irinotecan, isotretinoin, ixabepilone, ixempra, kidrolase (t), lanacort, lapatinib, l-asparaginase, lenalidomide, letrozole, leucovorin, leukeran, leukine, leuprolide, leurocristine, leustatin, liposomal ara-C, liquid pred, lomustine, L-sarcolysin, lupron, lupron depot, matulane, maxidex, mechlorethamine, mechlorethamine hydrochloride, medralone, medrol, megace, megestrol, megestrol acetate, melphalan, mercaptopurine, mesna, mesnex, methotrexate, methotrexate sodium, methylprednisolone, meticorten, mitomycin, mitomycin-c, mitoxantrone, m-prednisol, mustargen, mustine, mutamycin, myleran, mylocel, mylotarg, navelbine, nelarabine, neosar, neulasta, neumega, neupogen, nexavar, nilandron, nilutamide, nipent, nitrogen mustard, novaldex, novantrone, nplate, octreotide, octreotide acetate, oncospar, oncovin, ontak, onxal, oprelvekin, orapred, orasone, oxaliplatin, paclitaxel, paclitaxel protein-bound, pamidronate, panitumumab, panretin, paraplatin, pediapred, PEG Interferon, pegaspargase, pegfilgrastim, peginterferon alfa-2b, PEG-L-asparaginase, pemetrexed, pentostatin, phenylalanine mustard, platinol, platinol-AQ, prednisolone, prednisone, prelone, procarbazine, proleukin, prolifeprospan 20 with carmustine implant, purinethol, raloxifene, revlimid, rheumatrex, rituxan, rituximab, romiplostim, rubex, rubidomycin hydrochloride, sandostatin, sandostatin LAR, sargramostim, solu-cortef, solu-medrol, sorafenib, streptozocin, sunitinib, sutent, tamoxifen, tarceva, targretin, taxol, taxotere, temodar, temozolomide, temsirolimus, teniposide, thalidomide, thalomid, theracys, thioguanine, thioguanine tabloid, thiophosphoamide, thioplex, thiotepa, toposar, topotecan, toremifene, torisel, tositumomab, trastuzumab, treanda, tretinoin, trexall, trisenox, vectibix, velban, vepesid, vesanoid, viadur, vidaza, vinblastine, vinblastine sulfate, vincasar pfs, vincristine, vinorelbine, vinorelbine tartrate, vorinostat, vumon, xeloda, zanosar, zevalin, zinecard, zoladex, zoledronic acid, zolinza and zometa.

34. A method for treating and/or preventing a bone- or cartilage disorder, comprising administering to a patient in need of treatment, an effective amount of humanin or a humanin-like peptide (HLP) or a fragment, analog or derivative thereof.

35. A method for treating and/or preventing a drug-induced bone- or cartilage disorder, comprising administering to a patient in need of treatment, an effective amount of the peptide according to claim 34.

36. The method according to claim 34 or 35, wherein said peptide is selected from an amino acid sequence selected from SEQ ID NO:1 to SEQ ID NO:21, as well as an amino acid sequence having at least 60%, at least 65%, at least 70%, at least 75%, at least 80%, or at least 85% sequence identity to the peptide sequences defined in SEQ ID NO:1 to SEQ ID NO:21.

37. The method according to claim 34 or 35, wherein said peptide comprises an amino acid sequence selected from SEQ ID NO:1 to SEQ ID NO:21.

38. The method according to claim 34 or 35, wherein said peptide comprises an amino acid sequence of SEQ ID NO:2.

39. The method according to any of the claims 34 to 38, wherein said bone- or cartilage disorder is either primary or secondary bone- or cartilage disorder.

40. The method according to any of the claims 34 to 39, wherein said drug-induced bone or cartilage disorder is drug-induced by drugs used in the treatment of rheumatological disorders, respiratory disorders, gastrointestinal disorders, cardiovascular disorders, endocrinological disorders, cancer, neurodegenerative disorders, kidney disorders, liver disorders, dermatologic disorders, allergic disorders, inflammatory disorders, metabolic disorders, undesired immune response, undesired inflammatory response, obesity or diabetes.

41. The method according to any of the claims 34 to 40, wherein said drug-induced bone or cartilage disorder is selected from bone growth impairment, short stature and osteoporosis.

42. The method according to any of the claims 34 to 41, wherein said peptide improves bone growth.

43. The method according to any of the claims 34 to 41, wherein said peptide prevents development of osteoporosis.

44. The method according to claim 40, wherein said drug-induced bone or cartilage disorder is induced by an anti-inflammatory drug.

45. The method according to claim 44, wherein said anti-inflammatory drug is a glucocorticoid drug.

46. The method according to claim 45, wherein said glucocorticoid drug is selected from hydrocortisone, hydrocortisone buteprate, hydrocortisone butyrate, budesonide, ciclesonide, cortisone acetate, deflazacort, medrysone, tixocortol, cloprednol, halcinonide, pregnadiene, rimexolone, flunisolide, triamcinolone, amcinonide, fluocinolone acetonide, fluocinonide, fluorometholone, clocortolone, diflucortolone, fluocortin, desoximetasone, prednisone, prednisolone, methylprednisolone, methylprednisolone aceponate, prednicarbate, prednylidene, desonide, fluprednisolone, difluprednate, fluperolone, meprednisone, dexamethasone, betamethasone, triamcinolone, beclometasone, dobetasone, diflorasone, halometasone, ulobetasol, fludrocortisone acetate, beclomethasone dipropionate, beclomethasone monopropionate, paramethasone, alclometasone, fluclorolone, flumetasone, fluprednidene, triamcinolone, flunisolide, cortivazol, fluticasone, fluticasone propionate, fluticasone furoate, loteprednol, fludroxycortide, formocortal and mometasone furoate.

47. The method according to claim 45, wherein said glucocorticoid drug is dexamethasone.

48. The method according to claim 40, wherein said drug-induced bone or cartilage disorder is induced by an anti-cancer drug.

49. The method according to claim 48, wherein said anti-cancer drug is a proteasome inhibitor.

50. The method according to claim 49, wherein said proteasome inhibitor is selected from bortezomib, MLN9708, MLN273, MLN519, NPI-0052, disulfiram, ritonavir, lactacystin, CEP-18770, carfilzomib, PS-519, MG132, MG 262, ALLN, fellutamide B, tyropeptin A, omuralide, salinosporamide A, eponeomycine, epoxomicin, TMC-95A, syringolin A, glidobactin A, TMC-95 analogs, NLVS and ZLVS.

51. The method according to claim 49, wherein said proteasome inhibitor is bortezomib.

52. The method according to claim 40, wherein said drug-induced bone or cartilage disorder is induced by a GnRH agonist drug, such as leuprolide, buserelin, nafarelin, histrelin, goserelin or deslorelin.

53. The method according to claim 40, wherein said drug-induced bone or cartilage disorder is induced by a GnRH antagonist drug, such as cetrorelix, ganirelix, abarelix and degarelix.

54. The method for treating and/or preventing a bone- or cartilage disorder, according to any one of claims 34 to 53, wherein said peptide is administered in combination with a drug known to induce a bone- or cartilage disorder.

55. The method according to claim 54, wherein said drug is selected from an anti-inflammatory drug, a selective estrogen receptor modulator drug, an anti-androgen drug, an aromatase inhibitor drug and an anti-cancer drug.

56. The method according to claim 54, wherein said drug is an anti-inflammatory drug.

57. The method according to claim 56, wherein said anti-inflammatory drug is a glucocorticoid drug.

58. The method according to claim 57, wherein said glucocorticoid drug is selected from hydrocortisone, hydrocortisone buteprate, hydrocortisone butyrate, budesonide, ciclesonide, cortisone acetate, deflazacort, medrysone, tixocortol, cloprednol, halcinonide, pregnadiene, rimexolone, flunisolide, triamcinolone, amcinonide, fluocinolone acetonide, fluocinonide, fluorometholone, clocortolone, diflucortolone, fluocortin, desoximetasone, prednisone, prednisolone, methylprednisolone, methylprednisolone aceponate, prednicarbate, prednylidene, desonide, fluprednisolone, difluprednate, fluperolone, meprednisone, dexamethasone, betamethasone, triamcinolone, beclometasone, dobetasone, diflorasone, halometasone, ulobetasol, fludrocortisone acetate, beclomethasone dipropionate, beclomethasone monopropionate, paramethasone, alclometasone, fluclorolone, flumetasone, fluprednidene, triamcinolone, flunisolide, cortivazol, fluticasone, fluticasone propionate, fluticasone furoate, loteprednol, fludroxycortide, formocortal and mometasone furoate.

59. The method according to claim 57, wherein said glucocorticoid drug is dexamethasone.

60. The method according to claim 54, wherein said drug is an anti-cancer drug.

61. The method according to claim 60, wherein said anti-cancer drug is a proteasome inhibitor.

62. The method according to claim 61, wherein said proteasome inhibitor is selected from bortezomib, MLN9708, MLN273, MLN519, NPI-0052, disulfiram, ritonavir, lactacystin, CEP-18770, carfilzomib, PS-519, MG132, MG 262, ALLN, fellutamide B, tyropeptin A, omuralide, salinosporamide A, eponeomycine, epoxomicin, TMC-95A, syringolin A, glidobactin A, TMC-95 analogs, NLVS and ZLVS.

63. The method according to claim 61, wherein said proteasome inhibitor is bortezomib.

64. The method according to claim 54, wherein said drug is a GnRH agonist drug, such as leuprolide, buserelin, nafarelin, histrelin, goserelin or deslorelin.

65. The method according to claim 54, wherein said drug is a GnRH antagonist drug, such as cetrorelix, ganirelix, abarelix and degarelix.

66. The method according to claim 54, wherein said drug is selected from leuprolide, buserelin, nafarelin, histrelin, goserelin, deslorelin, cetrorelix, ganirelix, abarelix, degarelix, letrozole, anastrozole, exemestane, vorozole, formestahe, fadrozole, aminoglutethimide, testolactone, tamoxifen, afimoxifene, 4-hydroxytamoxifen, arzoxifene, bazedoxifene, clomifene, femarelle, lasofoxifene, ormeloxifene, raloxifene, tamoxifentoremifene, spironolactone, cyproterone acetate, flutamide, nilutamide, bicalutamide, ketoconazole, finasteride, dutasteride, cyclophosphamide, mechlorethamine, uramustine, melphalan, chlorambucil, ifosfamide, carmustine, lomustine, streptozocin, busulfan, thiotepa, cisplatin, carboplatin, nedaplatin, oxaliplatin, satraplatin, triplatin tetranitrate, procarbazine, altretamine, dacarbazine, mitozolomide, temozolomide, imatinib mesylate, erlotinib, gefitinib, sunitinib, roscovitine, bevacizumab, rapamycin, cyclosporin A, tacrolimus, 5-fluorouracil, methotrexate, etoposide, doxorubicin, actinomycine, vitamin A acid, 13-cis-retinoic acid, 2-chlorodeoxyadenosine, 5-azacitidine, 5-fluorouracil, 6-mercaptopurine, 6-thioguanine, abraxane, accutane, actinomycin-d, adriamycin, adrucil, afinitor, agrylin, ala-cort, aldesleukin, alemtuzumab, alitretinoin, alkaban-aq, alkeran, all-transretinoic acid, alpha interferon, altretamine, amethopterin, amifostine, aminoglutethimide, anagrelide, anandron, anastrozole, arabinosylcytosine, ara-c, aranesp, aredia, arimidex, aromasin, arranon, arsenic trioxide, asparaginase, avastin, azacitidine, bendamustine, bevacizumab, bexarotene, bicalutamide, blenoxane, bleomycin, busulfan, busulfex, calcium leucovorin, campath, camptosar, camptothecin-11, capecitabine, carac, carboplatin, carmustine, carmustine wafer, casodex, cerubidine, cetuximab, chlorambucil, cisplatin, citrovorum factor, cladribine, cortisone, cosmegen, cyclophosphamide, cytadren, cytarabine, cytarabine liposomal, cytosar-U, cytoxan, dacarbazine, dacogen, dactinomycin, darbepoetin alfa, dasatinib, daunomycin, daunorubicin, daunorubicin hydrochloride, daunorubicin liposomal, daunoxome, decadron, decitabine, delta-cortef, deltasone, denileukin diftitox, depocyt, dexamethasone acetate, dexamethasone sodium phosphate, dexasone, dexrazoxane, dhad, dic, diodex, docetaxel, doxil, doxorubicin, doxorubicin liposomal, droxia, duralone, efudex, eligard, ellence, eloxatin, elspar, emcyt, epirubicin, epoetin alfa, erbitux, erlotinib, erwinia L-asparaginase, estramustine, ethyol, etopophos, etoposide, etoposide phosphate, eulexin, everolimus, evista, exemestane, fareston, faslodex, femara, filgrastim, floxuridine, fludara, fludarabine, fluoroplex, fluorouracil, fluorouracil (cream), fluoxymesterone, flutamide, folinic acid, fulvestrant, gefitinib, gemcitabine, gemtuzumab ozogamicin, gemzar, gleevec, gliadel wafer, goserelin, halotestin, herceptin, hexadrol, hexalen, hexamethylmelamine, hycamtin, hydrea, hydrocort acetate, hydrocortisone, hydrocortisone sodium phosphate, hydrocortisone sodium succinate, hydrocortone phosphate, hydroxyurea, ibritumomab, ibritumomab tiuxetan, idamycin, idarubicin, ifex, ifosfamide, imatinib mesylate, imidazole carboxamide, interferon alfa, interleukin-2, interleukin-11, intron A (interferon alfa-2b), iressa, irinotecan, isotretinoin, ixabepilone, ixempra, kidrolase (t), lanacort, lapatinib, l-asparaginase, lenalidomide, letrozole, leucovorin, leukeran, leukine, leuprolide, leurocristine, leustatin, liposomal ara-C, liquid pred, lomustine, L-sarcolysin, lupron, lupron depot, matulane, maxidex, mechlorethamine, mechlorethamine hydrochloride, medralone, medrol, megace, megestrol, megestrol acetate, melphalan, mercaptopurine, mesna, mesnex, methotrexate, methotrexate sodium, methylprednisolone, meticorten, mitomycin, mitomycin-c, mitoxantrone, m-prednisol, mustargen, mustine, mutamycin, myleran, mylocel, mylotarg, navelbine, nelarabine, neosar, neulasta, neumega, neupogen, nexavar, nilandron, nilutamide, nipent, nitrogen mustard, novaldex, novantrone, nplate, octreotide, octreotide acetate, oncospar, oncovin, ontak, onxal, oprelvekin, orapred, orasone, oxaliplatin, paclitaxel, paclitaxel protein-bound, pamidronate, panitumumab, panretin, paraplatin, pediapred, PEG Interferon, pegaspargase, pegfilgrastim, peginterferon alfa-2b, PEG-L-asparaginase, pemetrexed, pentostatin, phenylalanine mustard, platinol, platinol-AQ, prednisolone, prednisone, prelone, procarbazine, proleukin, prolifeprospan 20 with carmustine implant, purinethol, raloxifene, revlimid, rheumatrex, rituxan, rituximab, romiplostim, rubex, rubidomycin hydrochloride, sandostatin, sandostatin LAR, sargramostim, solu-cortef, solu-medrol, sorafenib, streptozocin, sunitinib, sutent, tamoxifen, tarceva, targretin, taiiol, taxotere, temodar, temozolomide, temsirolimus, teniposide, thalidomide, thalomid, theracys, thioguanine, thioguanine tabloid, thiophosphoamide, thioplex, thiotepa, toposar, topotecan, toremifene, torisel, tositumomab, trastuzumab, treanda, tretinoin, trexall, trisenox, vectibix, velban, vepesid, vesanoid, viadur, vidaza, vinblastine, vinblastine sulfate, vincasar pfs, vincristine, vinorelbine, vinorelbine tartrate, vorinostat, vumon, xeloda, zanosar, zevalin, zinecard, zoladex, zoledronic acid, zolinza and zometa.

67. A pharmaceutical composition, comprising a peptide according to any of the claims 1 to 5, together with at least one pharmaceutically acceptable carrier or excipient, for use in the treatment and/or prevention of a disorder that negatively affects cartilage tissues or bone tissues.

68. A method of treating or preventing a bone- or cartilage disorder, comprising administering to a patient in need of treatment, an effective amount of an agent that regulates/affects the local production and/or expression of humanin or another peptide expressed by the humanin gene in cartilage tissues or bone.
